(19) United States
(12) Patent Application Publication
(10) Pub. No.: US 2010/0190755 A1
(43) Pub. Date: Jul. 29, 2010

(54) TETRACYCLINE COMPOUNDS FOR THE TREATMENT OF RHEUMATOID ARTHRITIS AND RELATED METHODS OF TREATMENT

(76) Inventors: Paul Abato, Providence, RI (US); Todd Bowser, Charlton, MA (US); Paul Higgins, Danvers, MA (US); Atul K. Verma, Mansfield, MA (US); Jie Zhang-Hoover, Tewksbury, MA (US)

Correspondence Address: Mintz Levin Cohn Ferris Glovsky and Popeo, P.C., One Financial Center, Boston, MA, 02111, US

(21) Appl. No.: 12/563,923

(22) Filed: Sep. 21, 2009

Related U.S. Application Data
(60) Provisional application No. 61/098,594, filed on Sep. 19, 2008.
(60) Provisional application No. 61/108,386, filed on Oct. 24, 2008.

(51) Int. Cl. A61K 31/65; A61P 19/02

(52) U.S. Cl. 514/152

(57) ABSTRACT

The present invention pertains, at least in part, to substituted tetracycline compounds. The present invention also pertains to methods for treating rheumatoid arthritis in a subject, comprising administering to the subject a tetracycline compound of the invention.

### RELATED APPLICATIONS

This application claims the benefit of priority under 35 U.S.C. 119(e) to pending U.S. Provisional Application No. 61/098,594, filed on Sep. 19, 2008, and pending U.S. Provisional Application No. 61/108,386, filed on Oct. 24, 2008, the entire contents of each are incorporated herein by reference.

### BACKGROUND OF THE INVENTION

The development of the tetracycline antibiotics has lead to several important compounds such as chlortetracycline, oxytetracycline, tetracycline, and minocycline.

Historically, soon after their initial development and introduction, the tetracyclines were found to be highly effective pharmacologically against rickettsiae; a number of gram-positive and gram-negative bacteria; and the agents responsible for lymphogranuloma venereum, inclusion conjunctivitis, and psittacosis. Hence, tetracyclines became known as “broad spectrum” antibiotics. With the subsequent establishment of their in vitro antimicrobial activity, effectiveness in experimental infections, and pharmacological properties, the tetracyclines as a class rapidly became widely used for therapeutic purposes. However, this widespread use of tetracyclines for both major and minor illnesses and diseases led directly to the emergence of resistance to these antibiotics even among highly susceptible bacterial species both commensal and pathogenic (e.g., pneumococci and Salmonella). The rise of tetracycline-resistant organisms has resulted in a general decline in use of tetracyclines and tetracycline analogue compositions as antibiotics of choice.

Rheumatoid arthritis (RA) is a chronic autoimmune condition that is characterized by synovial infiltration of activated inflammatory cells, synovial membrane hyperplasia, neoangiogenesis, and progressive destruction of cartilage and bone. Conventional first line therapy for rheumatoid arthritis includes nonsteroidal anti-inflammatory drugs (NSAIDs) followed by disease-modifying anti-rheumatic drugs (DMARDs), such as methotrexate and hydroxychloroquine. Minocycline has shown some beneficial effects in treating rheumatoid arthritis. A number of double-blind, placebo-controlled trials have concluded that early seropositive (<1 year of disease) rheumatoid arthritis patients respond positively to a 3-6 month minocycline treatment after 6 month, 1 year and 4 year follow-ups. However, long term use of minocycline would have undesirable consequences (e.g. gastrointestinal upset) due to its antibacterial activity.

Accordingly, it would be advantageous to develop substituted tetracycline compounds that are effective at treating rheumatoid arthritis and lack the antibacterial activity of previously known tetracycline compounds.

### SUMMARY OF THE INVENTION

The invention pertains, at least in part, to 7-substituted tetracycline compounds of Formula I:

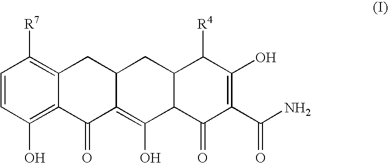

wherein:

R⁴ is amino or hydrogen; and

R⁷ is substituted or unsubstituted alkyl, substituted or unsubstituted heterocyclyl, substituted or unsubstituted phenyl, substituted or unsubstituted heteroaryl, or substituted or unsubstituted acyl;

or a pharmaceutically acceptable salt, ester or prodrug thereof.

The invention also pertains to 7-substituted 4-dedimethylamino sancycline compounds of the formula II-A:

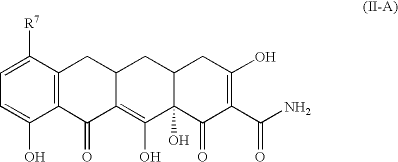

wherein:

R⁷ is substituted or unsubstituted C₁-C₅ alkyl, substituted or unsubstituted piperidinyl, substituted or unsubstituted phenyl, substituted or unsubstituted heteroaryl, or substituted or unsubstituted acyl;

or a pharmaceutically acceptable salt, ester or prodrug thereof.

The invention also pertains to 7-substituted sancycline compounds of the formula II-B:

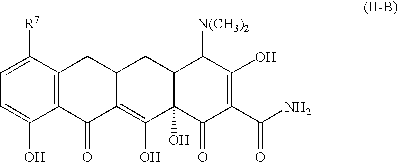

wherein:

R⁷ is substituted or unsubstituted C₁-C₅ alkyl, substituted or unsubstituted piperidinyl, substituted or unsubstituted phenyl, substituted or unsubstituted heteroaryl, or substituted or unsubstituted acyl;

or a pharmaceutically acceptable salt, ester or prodrug thereof.

The invention also pertains to 9-substituted tetracycline compounds of Formula III:

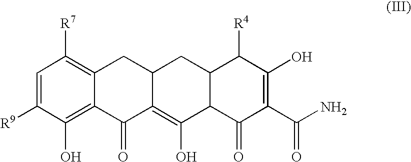

wherein:

R⁴ is amino or hydrogen;

R⁷ is amino or hydrogen; and

R⁹ is substituted or unsubstituted alkyl, substituted or unsubstituted heterocyclyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

The invention also pertains to 9-substituted 4-dedimethylamino minocycline compounds of Formula IV-A:

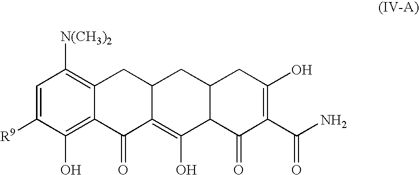

wherein:

R⁹ is substituted or unsubstituted C₁-C₅ alkyl, substituted or unsubstituted tetrahydropyranyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

The invention also pertains to 9-substituted minocycline compounds of Formula IV-B:

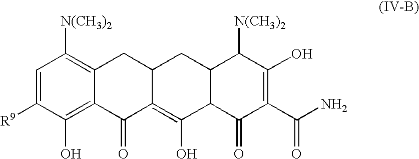

wherein:

R⁹ is substituted or unsubstituted C₁-C₅ alkyl, substituted or unsubstituted tetrahydropyranyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

The invention also pertains to 7,9-disubstituted tetracycline compounds of formula V:

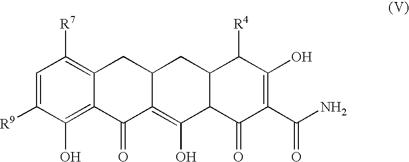

wherein:

R⁴ is amino or hydrogen;

R⁷ is substituted or unsubstituted C₁-C₅ alkyl, substituted or unsubstituted heterocyclyl, substituted or unsubstituted phenyl, substituted or unsubstituted heteroaryl, substituted or unsubstituted acyl; and

R⁹ is substituted or unsubstituted C₁-C₅ alkyl, substituted or unsubstituted heterocyclyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

The invention also pertains to 10-substituted tetracycline compounds of formula VI:

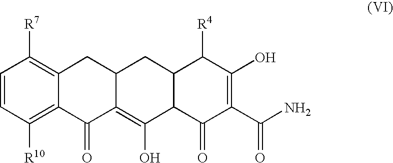

wherein:

R⁴ is amino or hydrogen;

R⁷ amino or hydrogen; and

R¹⁰ is hydrogen, substituted or unsubstituted C₁-C₅ alkyl, substituted or unsubstituted heterocyclyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

The invention also pertains to a method for treating rheumatoid arthritis in a subject, comprising administering to the subject a tetracycline compound of the invention (e.g., of Formula I, II-A, II-B, III, IV-A, IV-B, V, VI or Table 2), such that the rheumatoid arthritis is treated. In one embodiment, the tetracycline compound does not exhibit antibacterial activity.

In one embodiment, the present invention provides a method for treating rheumatoid arthritis in a subject, comprising administering to the subject a tetracycline compound of Formula I:

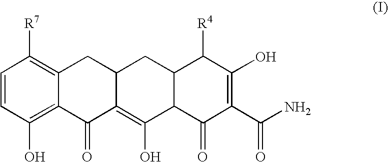

wherein:

R⁴ is amino or hydrogen; and

R⁷ is substituted or unsubstituted alkyl, substituted or unsubstituted heterocyclyl, substituted or unsubstituted phenyl, substituted or unsubstituted heteroaryl, or substituted or unsubstituted acyl;

or a pharmaceutically acceptable salt, ester or prodrug thereof; such that the rheumatoid arthritis is treated in the subject.

In another embodiment, the present invention provides a method for treating rheumatoid arthritis in a subject, comprising administering to the subject a tetracycline compound of Formula II-A:

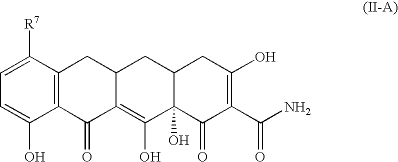

wherein:

R⁷ is substituted or unsubstituted C₁-C₅ alkyl, substituted or unsubstituted piperidinyl, substituted or unsubstituted phenyl, substituted or unsubstituted heteroaryl, or substituted or unsubstituted acyl;

or a pharmaceutically acceptable salt, ester or prodrug thereof; such that the rheumatoid arthritis is treated in the subject.

In another embodiment, the present invention provides a method for treating rheumatoid arthritis in a subject, comprising administering to the subject a tetracycline compound of Formula II-B:

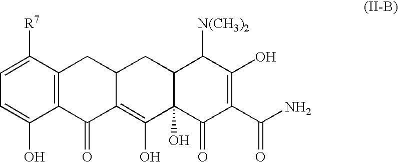

wherein:

R⁷ is substituted or unsubstituted C₁-C₅ alkyl, substituted or unsubstituted piperidinyl, substituted or unsubstituted phenyl, substituted or unsubstituted heteroaryl, or substituted or unsubstituted acyl;

or a pharmaceutically acceptable salt, ester or prodrug thereof; such that the rheumatoid arthritis is treated in the subject.

In another embodiment, the present invention provides a method for treating rheumatoid arthritis in a subject, comprising administering to the subject a tetracycline compound of Formula III:

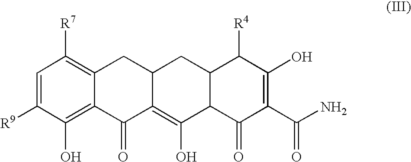

wherein:

R⁴ is amino or hydrogen;

R⁷ is amino or hydrogen; and

R⁹ is substituted or unsubstituted alkyl, substituted or unsubstituted heterocyclyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof; such that the rheumatoid arthritis is treated in the subject.

In another embodiment, the present invention provides a method for treating rheumatoid arthritis in a subject, comprising administering to the subject a tetracycline compound of Formula IV-A:

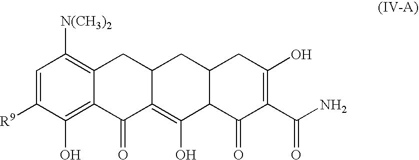

wherein:

R⁹ is substituted or unsubstituted C₁-C₅ alkyl, substituted or unsubstituted tetrahydropyranyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof; such that the rheumatoid arthritis is treated in the subject.

In another embodiment, the present invention provides a method for treating rheumatoid arthritis in a subject, comprising administering to the subject a tetracycline compound of Formula IV-B:

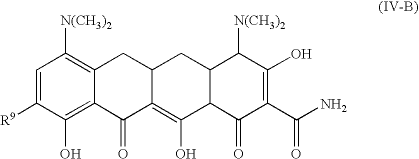

wherein:

R⁹ is substituted or unsubstituted C₁-C₅ alkyl, substituted or unsubstituted tetrahydropyranyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof; such that the rheumatoid arthritis is treated in the subject.

In another embodiment, the present invention provides a method for treating rheumatoid arthritis in a subject, comprising administering to the subject a tetracycline compound of Formula V:

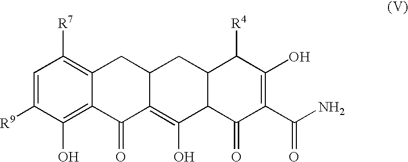

wherein:

R⁴ is amino or hydrogen;

R⁷ is substituted or unsubstituted C₁-C₅ alkyl, substituted or unsubstituted heterocyclyl, substituted or unsubstituted phenyl, substituted or unsubstituted heteroaryl, substituted or unsubstituted acyl; and

R⁹ is substituted or unsubstituted C₁-C₅ alkyl, substituted or unsubstituted heterocyclyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof; such that the rheumatoid arthritis is treated in the subject.

In another embodiment, the present invention provides a method for treating rheumatoid arthritis in a subject, comprising administering to the subject a tetracycline compound of Formula VI:

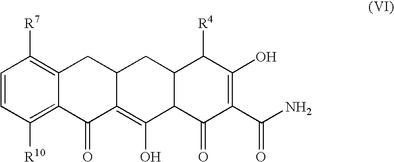

wherein:

R⁴ is amino or hydrogen;

R⁷ amino or hydrogen; and

R¹⁰ is hydrogen, substituted or unsubstituted C₁-C₅ alkyl, substituted or unsubstituted heterocyclyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof; such that the rheumatoid arthritis is treated in the subject.

The invention also includes pharmaceutical compositions comprising an effective amount of a tetracycline compound of the invention (e.g., of Formula I, II-A, II-B, III, IV-A, IV-B, V, VI or Table 2), and, optionally, a pharmaceutically acceptable carrier.

### DETAILED DESCRIPTION OF THE INVENTION

The present invention provides, in part, a method for the treatment of rheumatoid arthritis (RA). Such a method can include, but is not limited to, the administration of orally available modulators of T-Cell activation and inhibitors of downstream effects.

The present invention pertains, at least in part, to modified tetracycline compounds. These tetracycline compounds can be used to treat rheumatoid arthritis as well as other known applications for minocycline and tetracycline compounds in general, such as blocking tetracycline efflux and modulation of gene expression.

The term “tetracycline compound” includes many compounds with a similar ring structure to tetracycline. Examples of tetracycline compounds include: tetracycline, chlortetracycline, oxytetracycline, demeclocycline, methacycline, sancycline, doxycycline, and minocycline. Other derivatives and analogues comprising a similar four ring structure are also included. The term also includes 4-dedimethylamino tetracycline compounds. Table 1 depicts tetracycline and several known tetracycline derivatives.

TABLE 1

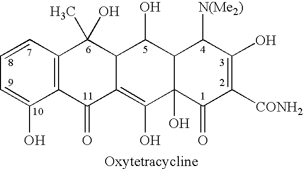

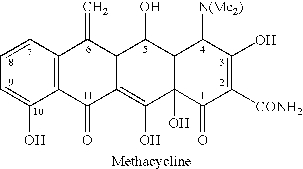

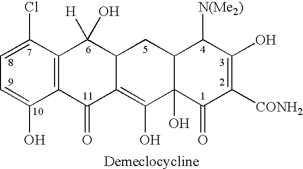

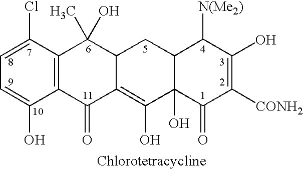

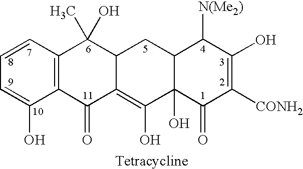

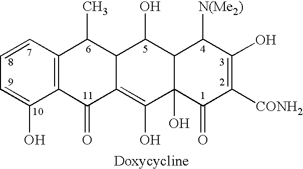

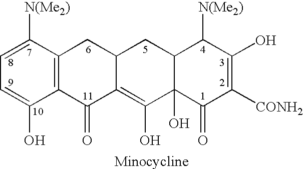

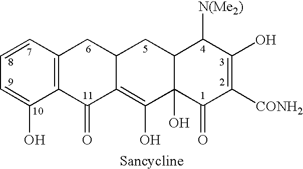

### I. 7-Substituted Tetracycline Compounds

The term “7-substituted tetracycline compounds” includes tetracycline compounds with a substitution at the 7-position. In one embodiment, the substitution at the 7-position enhances the ability of the tetracycline compound to perform its intended function, e.g., to treat rheumatoid arthritis. In an embodiment, the 7-substituted tetracycline compound is 7-substituted sancycline (i.e., wherein R⁴ is dimethylamino). In another embodiment, the 7-substituted tetracycline compound is 7-substituted 4-dedimethylamino sancycline (i.e., wherein R⁴ is hydrogen).

The invention pertains to 7-substituted tetracycline compounds of Formula I:

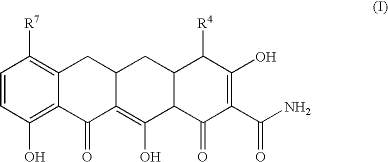

wherein:

R⁴ is amino or hydrogen; and

R⁷ is substituted or unsubstituted alkyl, substituted or unsubstituted heterocyclyl, substituted or unsubstituted phenyl, substituted or unsubstituted heteroaryl, or substituted or unsubstituted acyl;

or a pharmaceutically acceptable salt, ester or prodrug thereof.

In an embodiment, R⁴ is a dialkylamino group (e.g., dimethylamino).

In another embodiment, R⁷ is substituted or unsubstituted heteroaryl. In another embodiment, R⁷ is substituted or unsubstituted phenyl. The phenyl R⁷ group or the heteroaryl R⁷ group can be substituted with any substituent which allows the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In a further embodiment, the phenyl R⁷ group or the heteroaryl R⁷ group is substituted with substituted or unsubstituted alkyl. Examples of substituents of the alkyl include heterocycles such as, morpholine, piperidine, and pyrrolidine. In another further embodiment, the phenyl R⁷ group or the heteroaryl R⁷ group is substituted with an amino group. The amino group also may be further substituted e.g., with an alkyl, alkenyl, alkynyl, carbonyl, alkoxy or aryl (e.g., substituted or unsubstituted, heteroaryl, phenyl, etc.) group. The amino substituent may be substituted with any substituent or combination of substituents which allow it to perform its intended function. Examples of such substituents include, but are not limited to, halogens (e.g., fluorine, chlorine, bromine, iodine, etc.), amino (e.g., which can in turn be substituted with an alkyl, carbonyl, alkenyl, alkynyl, or aryl moiety), and arylamino (e.g., phenylamino).

The phenyl R⁷ group or the heteroaryl R⁷ group may also be substituted with alkoxy groups. Examples of alkoxy groups include, but are not limited to, methoxy, ethoxy, propoxy, butoxy, perfluoromethoxy, perchloromethoxy, methylenedioxy, etc. The phenyl group or the heteroaryl group may also be substituted with an amide group such as a carbamate moiety (e.g., an alkoxycarbonylamino group).

The heteroaryl R⁷ group also may be substituted or unsubstituted biaryl, e.g., naphthyl, fluorenyl, etc. The biaryl R⁷ group can be substituted with any substituent which allow it to perform its intended function. Examples of substituents include but are not limited to, alkyl, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In an embodiment, R⁷ is a heteroaryl group substituted with amino or formyl.

Examples of heteroaryl R⁷ moieties include, but are not limited to, furanyl, imidazolyl, benzothiophenyl, benzofuranyl, quinolinyl, isoquinolinyl, pyridinyl, pyrazolyl, benzodioxazolyl, benzoxazolyl, thiofuranyl, oxadiazolyl, pyrrolyl, benzothiazolyl, benzoimidazolyl, indolyl, thienyl, pyrimidyl, pyrazinyl, purinyl, pyrazolyl, oxazolyl, isooxazolyl, naphthridinyl, thiazolyl, isothiazolyl, and deazapurinyl. In certain embodiments, the heteroaryl R⁷ group is oxazolyl.

In another embodiment, R⁷ is substituted or unsubstituted alkyl. The alkyl group can be a straight or branched chain, e.g., methyl, ethyl, i-propyl, n-propyl, n-butyl, i-butyl, t-butyl, pentyl, hexyl. etc. The alkyl group may also comprise a ring, e.g., a cycloalkyl (e.g., cyclopentyl, cyclohexyl, cyclopropyl, or cyclobutyl). The alkyl R⁷ group may be substituted with any substituent or combination of substituents which allows the compound to perform its intended function. Examples of substituents include, but are not limited to, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In certain embodiments, the alkyl group is substituted with an amino, hydroxy, carboxy, carbonyl (e.g., substituted carbonyl), heterocyclic or aryl groups. Examples of heterocyclic or aryl groups include, for example, furanyl, imidazolyl, benzothiophenyl, benzofuranyl, quinolinyl, isoquinolinyl, benzodioxazolyl, benzoxazolyl, benzothiazolyl, benzoimidazolyl, methylenedioxyphenyl, indolyl, thienyl, pyridinyl, pyrazolyl, pyrimidyl, pyrazinyl, purinyl, pyrazolyl, oxazolyl, isooxazolyl, naphthridinyl, thiazolyl, isothiazolyl, and deazapurinyl. In a further embodiment, the aryl group is pyridinyl.

In another embodiment, R⁷ is substituted or unsubstituted heterocyclyl. The heterocyclyl R⁷ group can be substituted with any substituent which allow the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

Examples of heterocyclyl R⁷ moieties include, but are not limited to, pyrrolidinyl, dioxolanyl, imidazolidinyl, pyrazolidinyl, piperidinyl, dioxanyl, morpholinyl, dithianyl, thiomorpholinyl, piperazinyl and trithianyl. In one embodiment, the heterocyclyl R⁷ group is piperidinyl. In another embodiments, the heterocyclyl R⁷ group is tetrahydropyran. In another embodiment, the heterocyclyl moieties are saturated. In another embodiment, the heterocyclyl moieties are partially unsaturated.

In another embodiment, R⁷ is substituted or unsubstituted acyl. The acyl R⁷ group can be substituted with any substituent which allow the tetracycline compound to perform its intended function. Examples of substituents include, but are not limited to, alkyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. In a further embodiment, R⁷ is acetyl.

The invention also pertains to 7-substituted 4-dedimethylamino sancycline compounds of the formula II-A:

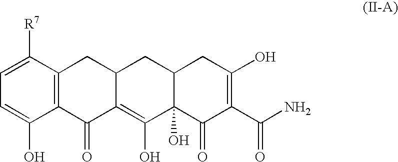

wherein:

R⁷ is substituted or unsubstituted C₁-C₅ alkyl, substituted or unsubstituted piperidinyl, substituted or unsubstituted phenyl, substituted or unsubstituted heteroaryl, or substituted or unsubstituted acyl;

or a pharmaceutically acceptable salt, ester or prodrug thereof.

The invention also pertains to 7-substituted sancycline compounds of the formula II-B:

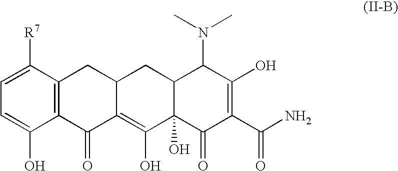

wherein:

R⁷ is substituted or unsubstituted C₁-C₅ alkyl, substituted or unsubstituted piperidinyl, substituted or unsubstituted phenyl, substituted or unsubstituted heteroaryl, or substituted or unsubstituted acyl;

or a pharmaceutically acceptable salt, ester or prodrug thereof.

The invention also pertains to the 7-substituted tetracycline compounds shown in Table 2, such as compounds B, C, D, L, N, AQ, BA, BB and BC.

Also included are pharmaceutically acceptable salts, esters and prodrugs of the compounds of formulae I, II-A, II-B and those shown in Table 2.

### II. 9-Substituted Tetracycline Compounds

The term “9-substituted tetracycline compounds” includes tetracycline compounds with a substitution at the 9-position. In one embodiment, the substitution at the 9-position enhances the ability of the tetracycline compound to perform its intended function, e.g., to treat rheumatoid arthritis. In an embodiment, the 9-substituted tetracycline compound is 9-substituted 4-dedimethylamino minocycline (i.e., wherein R⁴ is hydrogen and R⁷ is dimethylamino). In another embodiment, the 9-substituted tetracycline compound is 9-substituted minocycline (i.e., wherein R⁴ and R⁷ are each dimethylamino). In another embodiment, the 9-substituted tetracycline compound is 9-substituted doxycycline. In another embodiment, the 9-substituted tetracycline compound is 9-substituted 4-dedimethylamino doxycycline.

The invention also pertains to 9-substituted tetracycline compounds of Formula III:

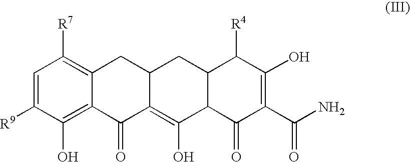

wherein:

R⁴ is amino or hydrogen;

R⁷ is amino or hydrogen; and

R⁹ is substituted or unsubstituted alkyl, substituted or unsubstituted heterocyclyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

In an embodiment, R⁴ is a dialkylamino group (e.g., dimethylamino). In another embodiment, R⁷ is a dialkylamino group (e.g., dimethylamino). In another embodiment, R⁴ and R⁷ are each dimethylamino.

In another embodiment, R⁹ is a substituted or unsubstituted heteroaryl group. In another embodiment, R⁹ is a substituted or unsubstituted phenyl group. The heteroaryl R⁹ group or the phenyl R⁹ group can be substituted with any substituent which allows the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In a further embodiment, the phenyl R⁹ group or the heteroaryl R⁹ group is substituted with substituted or unsubstituted alkyl. Examples of substituents of the alkyl include heterocycles such as, morpholine, piperidine, and pyrrolidine. In another further embodiment, the phenyl R⁹ group or the heteroaryl R⁹ group is substituted with an amino group. The amino group also may be further substituted e.g., with an alkyl, alkenyl, alkynyl, carbonyl, alkoxy or aryl (e.g., substituted or unsubstituted, heteroaryl, phenyl, etc.) group. The amino substituent may be substituted with any substituent or combination of substituents which allow it to perform its intended function. Examples of such substituents include halogens (e.g., fluorine, chlorine, bromine, iodine, etc.), amino (e.g., which can in turn be substituted with an alkyl, carbonyl, alkenyl, alkynyl, or aryl moiety), and arylamino (e.g., phenylamino).

The phenyl R⁹ group or the heteroaryl R⁹ group may also be substituted with alkoxy groups. Examples of alkoxy groups include, but are not limited to, methoxy, ethoxy, propoxy, butoxy, perfluoromethoxy, perchloromethoxy, methylenedioxy, etc. The phenyl group or the heteroaryl group may also be substituted with an amide group such as a carbamate moiety (e.g., an alkoxycarbonylamino group).

The heteroaryl R⁹ group also may be substituted or unsubstituted biaryl, e.g., naphthyl, fluorenyl, etc. The biaryl R⁹ group can be substituted with any substituent which allow it to perform its intended function. Examples of substituents include but are not limited to, alkyl, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In an embodiment, R⁹ is a heteroaryl group substituted with amino or formyl.

Examples of heteroaryl R⁹ moieties include, but are not limited to, furanyl, imidazolyl, benzothiophenyl, benzofuranyl, quinolinyl, isoquinolinyl, pyridinyl, pyrazolyl, benzodioxazolyl, benzoxazolyl, benzothiazolyl, benzoimidazolyl, indolyl, thienyl, pyrimidyl, thiofuranyl, oxadiazolyl, pyrrolyl, pyrazinyl, purinyl, pyrazolyl, oxazolyl, isoxazolyl, naphthridinyl, thiazolyl, isothiazolyl, and deazapurinyl. In certain embodiment, the heteroaryl R⁹ group is oxazolyl, thiofuranyl, isoxazolyl, pyrazolyl, pyridinyl, furanyl, thiazolyl, oxadiazolyl or pyrrolyl.

In another embodiment, R⁹ is substituted or unsubstituted alkyl. The alkyl group can be a straight or branched chain, e.g., methyl, ethyl, i-propyl, n-propyl, n-butyl, i-butyl, t-butyl, pentyl, hexyl. etc. The alkyl group may also comprise a ring, e.g., a cycloalkyl (e.g., cyclopentyl, cyclohexyl, cyclopropyl, or cyclobutyl). The alkyl R⁹ group may be substituted with any substituent or combination of substituents which allows the compound to perform its intended function. Examples of substituents include, but are not limited to, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In certain embodiments, the alkyl group is substituted with an amino, hydroxy, carboxy, carbonyl (e.g., substituted carbonyl), heterocyclic or aryl groups. Examples of heterocyclic or aryl groups include, for example, furanyl, imidazolyl, benzothiophenyl, benzofuranyl, quinolinyl, isoquinolinyl, benzodioxazolyl, benzoxazolyl, benzothiazolyl, benzoimidazolyl, indolyl, thienyl, pyridinyl, pyrazolyl, pyrimidyl, pyrazinyl, purinyl, pyrazolyl, oxazolyl, isooxazolyl, naphthridinyl, thiazolyl, isothiazolyl, and deazapurinyl. In a further embodiment, the aryl group is pyridinyl.

In another embodiment, R⁹ is substituted or unsubstituted heterocyclyl. The heterocyclyl R⁹ group can be substituted with any substituent which allow the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

Examples of heterocyclyl R⁹ moieties include, but are not limited to, pyrrolidinyl, dioxolanyl, imidazolidinyl, pyrazolidinyl, piperidinyl, dioxanyl, morpholinyl, dithianyl, thiomorpholinyl, piperazinyl and trithianyl. In one embodiment, the heterocyclyl R⁹ group is piperidinyl. In another embodiments, the heterocyclyl R⁹ group is tetrahydropyran. In another embodiment, the heterocyclyl moieties are saturated. In another embodiment, the heterocyclyl moieties are partially unsaturated.

In another embodiment, R⁹ is substituted or unsubstituted acyl. The acyl R⁹ group can be substituted with any substituent which allow the tetracycline compound to perform its intended function. Examples of substituents include, but are not limited to, alkyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. In a further embodiment, R⁹ is acetyl.

In another embodiment, R⁹ is substituted or unsubstituted imine. The imine R⁹ group can be substituted with any substituent which allow the tetracycline compound to perform its intended function. Examples of substituents include, but are not limited to, alkyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The invention also pertains to 9-substituted 4-dedimethylamino minocycline compounds of Formula IV-A:

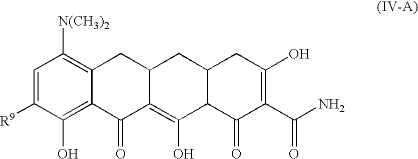

wherein:

R⁹ is substituted or unsubstituted C₁-C₅ alkyl, substituted or unsubstituted tetrahydropyranyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

The invention also pertains to 9-substituted minocycline compounds of Formula IV-B:

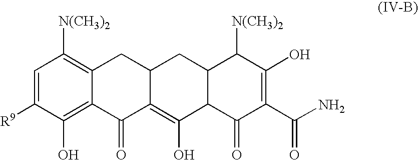

wherein:

R⁹ is substituted or unsubstituted C₁-C₅ alkyl, substituted or unsubstituted tetrahydropyranyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

The invention also pertains to the 9-substituted tetracycline compounds shown in Table 2, such as compounds A, E, G, H, I, J, K, M, O, P, R, S, T, U, V, W, X, Y, Z, AA, AB, AC, AD, AE, AF, AG, AH, AI, AJ, AK, AL, AM, AN, AO, AP, AR, AS, AT, AU, AV, AW, AX, AZ and BD.

Also included are pharmaceutically acceptable salts, esters and prodrugs of the compounds of formulae III, IV-A, IV-B and those shown in Table 2.

### III. 7,9-Disubstituted Tetracycline Compounds

The term “7,9-disubstituted tetracycline compounds” includes tetracycline compounds with substitution at the 7- and 9-positions. In one embodiment, the substitutions at the 7- and 9-positions enhances the ability of the tetracycline compound to perform its intended function, e.g., to treat rheumatoid arthritis. In an embodiment, the 7,9-disubstituted tetracycline compound is 7,9-disubstituted sancycline. In another embodiment, the 7,9-substituted tetracycline compound is 7,9-disubstituted 4-dedimethylamino sancycline. In another embodiment, the 7,9-disubstituted tetracycline compound is 7,9-disubstituted doxycycline. In another embodiment, the 7,9-disubstituted tetracycline compound is 7,9-disubstituted 4-dedimethylamino doxycycline.

The invention also pertains to 7,9-disubstituted tetracycline compounds of formula V:

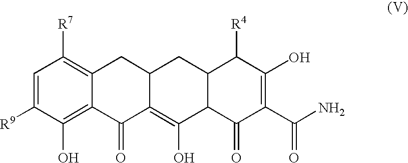

wherein:

R⁴ is amino or hydrogen;

R⁷ is substituted or unsubstituted C₁-C₅ alkyl, substituted or unsubstituted heterocyclyl, substituted or unsubstituted phenyl, substituted or unsubstituted heteroaryl, substituted or unsubstituted acyl; and

R⁹ is substituted or unsubstituted C₁-C₅ alkyl, substituted or unsubstituted heterocyclyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

In an embodiment, R⁴ is a dialkylamino group (e.g., dimethylamino).

In another embodiment, R⁷ is substituted or unsubstituted heteroaryl. In another embodiment, R⁷ is substituted or unsubstituted phenyl. The phenyl R⁷ group or the heteroaryl R⁷ group can be substituted with any substituent which allows the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In a further embodiment, the phenyl R⁷ group or the heteroaryl R⁷ group is substituted with substituted or unsubstituted alkyl. Examples of substituents of the alkyl include heterocycles such as, morpholine, piperidine, and pyrrolidine. In another further embodiment, the phenyl R⁷ group or the heteroaryl R⁷ group is substituted with an amino group. The amino group also may be further substituted e.g., with an alkyl, alkenyl, alkynyl, carbonyl, alkoxy or aryl (e.g., substituted or unsubstituted, heteroaryl, phenyl, etc.) group. The amino substituent may be substituted with any substituent or combination of substituents which allow it to perform its intended function. Examples of such substituents include halogens (e.g., fluorine, chlorine, bromine, iodine, etc.), amino (e.g., which can in turn be substituted with an alkyl, carbonyl, alkenyl, alkynyl, or aryl moiety), and arylamino (e.g., phenylamino).

The phenyl R⁷ group or the heteroaryl R⁷ group may also be substituted with alkoxy groups. Examples of alkoxy groups include, but are not limited to, methoxy, ethoxy, propoxy, butoxy, perfluoromethoxy, perchloromethoxy, methylenedioxy, etc. The phenyl group or the heteroaryl group may also be substituted with an amide group such as a carbamate moiety (e.g., an alkoxycarbonylamino group).

The heteroaryl R⁷ group also may be substituted or unsubstituted biaryl, e.g., naphthyl, fluorenyl, etc. The biaryl R⁷ group can be substituted with any substituent which allow it to perform its intended function. Examples of substituents include but are not limited to, alkyl, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In an embodiment, R⁷ is a heteroaryl group substituted with amino or formyl.

Examples of heteroaryl R⁷ moieties include, but are not limited to, furanyl, imidazolyl, benzothiophenyl, benzofuranyl, quinolinyl, isoquinolinyl, pyridinyl, pyrazolyl, benzodioxazolyl, benzoxazolyl, benzothiazolyl, benzoimidazolyl, thiofuranyl, oxadiazolyl, pyrrolyl, indolyl, thienyl, pyrimidyl, pyrazinyl, purinyl, pyrazolyl, oxazolyl, isooxazolyl, naphthridinyl, thiazolyl, isothiazolyl, and deazapurinyl. In certain embodiments, the heteroaryl R⁷ group is oxazolyl.

In another embodiment, R⁷ is substituted or unsubstituted alkyl. The alkyl group can be a straight or branched chain, e.g., methyl, ethyl, i-propyl, n-propyl, n-butyl, i-butyl, t-butyl, pentyl, hexyl. etc. The alkyl group may also comprise a ring, e.g., a cycloalkyl (e.g., cyclopentyl, cyclohexyl, cyclopropyl, or cyclobutyl). The alkyl R⁷ group may be substituted with any substituent or combination of substituents which allows the compound to perform its intended function. Examples of substituents include, but are not limited to, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In certain embodiments, the alkyl group is substituted with an amino, hydroxy, carboxy, carbonyl (e.g., substituted carbonyl), heterocyclic or aryl groups. Examples of heterocyclic or aryl groups include, for example, furanyl, imidazolyl, benzothiophenyl, benzofuranyl, quinolinyl, isoquinolinyl, benzodioxazolyl, benzoxazolyl, benzothiazolyl, benzoimidazolyl, methylenedioxyphenyl, indolyl, thienyl, pyridinyl, pyrazolyl, pyrimidyl, pyrazinyl, purinyl, pyrazolyl, oxazolyl, isooxazolyl, naphthridinyl, thiazolyl, isothiazolyl, and deazapurinyl. In a further embodiment, the aryl group is pyridinyl.

In another embodiment, R⁷ is substituted or unsubstituted heterocyclyl. The heterocyclyl R⁷ group can be substituted with any substituent which allow the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

Examples of heterocyclyl R⁷ moieties include, but are not limited to, pyrrolidinyl, dioxolanyl, imidazolidinyl, pyrazolidinyl, piperidinyl, dioxanyl, morpholinyl, dithianyl, thiomorpholinyl, piperazinyl and trithianyl. In one embodiment, the heterocyclyl R⁷ group is piperidinyl. In another embodiments, the heterocyclyl R⁷ group is tetrahydropyran. In another embodiment, the heterocyclyl moieties are saturated. In another embodiment, the heterocyclyl moieties are partially unsaturated.

In another embodiment, R⁷ is substituted or unsubstituted acyl. The acyl R⁷ group can be substituted with any substituent which allow the tetracycline compound to perform its intended function. Examples of substituents include, but are not limited to, alkyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. In a further embodiment, R⁷ is acetyl.

In one embodiment, R⁹ is a substituted or unsubstituted heteroaryl group. In another embodiment, R⁹ is a substituted or unsubstituted phenyl group. The heteroaryl R⁹ group or the phenyl R⁹ group can be substituted with any substituent which allow the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In a further embodiment, the phenyl R⁹ group or the heteroaryl R⁹ group is substituted with substituted or unsubstituted alkyl. Examples of substituents of the alkyl include heterocycles such as, morpholine, piperidine, and pyrrolidine. In another further embodiment, the phenyl R⁹ group or the heteroaryl R⁹ group is substituted with an amino group. The amino group also may be further substituted e.g., with an alkyl, alkenyl, alkynyl, carbonyl, alkoxy or aryl (e.g., substituted or unsubstituted, heteroaryl, phenyl, etc.) group. The amino substituent may be substituted with any substituent or combination of substituents which allow it to perform its intended function. Examples of such substituents include halogens (e.g., fluorine, chlorine, bromine, iodine, etc.), amino (e.g., which can in turn be substituted with an alkyl, carbonyl, alkenyl, alkynyl, or aryl moiety), and arylamino (e.g., phenylamino).

The phenyl R⁹ group or the heteroaryl R⁹ group may also be substituted with alkoxy groups. Examples of alkoxy groups include, but are not limited to, methoxy, ethoxy, propoxy, butoxy, perfluoromethoxy, perchloromethoxy, methylenedioxy, etc. The phenyl group or the heteroaryl group may also be substituted with an amide group such as a carbamate moiety (e.g., an alkoxycarbonylamino group).

The heteroaryl R⁹ group also may be substituted or unsubstituted biaryl, e.g., naphthyl, fluorenyl, etc. The biaryl R⁹ group can be substituted with any substituent which allow it to perform its intended function. Examples of substituents include but are not limited to, alkyl, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In an embodiment, R⁹ is a heteroaryl group substituted with amino or formyl.

Examples of heteroaryl R⁹ moieties include, but are not limited to, furanyl, imidazolyl, benzothiophenyl, benzofuranyl, quinolinyl, isoquinolinyl, pyridinyl, pyrazolyl, benzodioxazolyl, benzoxazolyl, benzothiazolyl, benzoimidazolyl, thiofuranyl, oxadiazolyl, pyrrolyl, indolyl, thienyl, pyrimidyl, pyrazinyl, purinyl, pyrazolyl, oxazolyl, isooxazolyl, naphthridinyl, thiazolyl, isothiazolyl, and deazapurinyl. In certain embodiments, the heteroaryl R⁹ group is oxazolyl, thiofuranyl, isoxazolyl, pyrazolyl, pyridinyl, furanyl, thiazolyl, oxadiazolyl or pyrrolyl.

In another embodiment, R⁹ is substituted or unsubstituted alkyl. The alkyl group can be a straight or branched chain, e.g., methyl, ethyl, i-propyl, n-propyl, n-butyl, i-butyl, t-butyl, pentyl, hexyl. etc. The alkyl group may also comprise a ring, e.g., a cycloalkyl (e.g., cyclopentyl, cyclohexyl, cyclopropyl, or cyclobutyl). The alkyl R⁹ group may be substituted with any substituent or combination of substituents which allows the compound to perform its intended function. Examples of substituents include, but are not limited to, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In certain embodiments, the alkyl group is substituted with an amino, hydroxy, carboxy, carbonyl (e.g., substituted carbonyl), heterocyclic or aryl groups. Examples of heterocyclic or aryl groups include, for example, furanyl, imidazolyl, benzothiophenyl, benzofuranyl, quinolinyl, isoquinolinyl, benzodioxazolyl, benzoxazolyl, benzothiazolyl, benzoimidazolyl, indolyl, thienyl, pyridinyl, pyrazolyl, pyrimidyl, pyrazinyl, purinyl, pyrazolyl, oxazolyl, isooxazolyl, naphthridinyl, thiazolyl, isothiazolyl, and deazapurinyl. In a further embodiment, the aryl group is pyridinyl.

In another embodiment, R⁹ is substituted or unsubstituted heterocyclyl. The heterocyclyl R⁹ group can be substituted with any substituent which allow the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

Examples of heterocyclyl R⁹ moieties include, but are not limited to, pyrrolidinyl, dioxolanyl, imidazolidinyl, pyrazolidinyl, piperidinyl, dioxanyl, morpholinyl, dithianyl, thiomorpholinyl, piperazinyl and trithianyl. In one embodiment, the heterocyclyl R⁹ group is piperidinyl. In another embodiments, the heterocyclyl R⁹ group is tetrahydropyran. In another embodiment, the heterocyclyl moieties are saturated. In another embodiment, the heterocyclyl moieties are partially unsaturated.

In another embodiment, R⁹ is substituted or unsubstituted acyl. The acyl R⁹ group can be substituted with any substituent which allow the tetracycline compound to perform its intended function. Examples of substituents include, but are not limited to, alkyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. In a further embodiment, R⁹ is acetyl.

In another embodiment, R⁹ is substituted or unsubstituted imine. The imine R⁹ group can be substituted with any substituent which allow the tetracycline compound to perform its intended function. Examples of substituents include, but are not limited to, alkyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

In one embodiment, R⁹ is not unsubstituted phenyl when R⁷ is unsubstituted phenyl.

Also included are pharmaceutically acceptable salts, esters and prodrugs of the compounds of formulae V.

### IV. 10-Substituted Tetracycline Compounds

In another embodiment, the 10-substituted tetracycline compound is a 10-substituted minocycline derivative. In one embodiment, the substitution at the 10-position enhances the ability of the tetracycline compound to perform its intended function, e.g., to treat rheumatoid arthritis. In another embodiment, the 10-substituted tetracycline compound is a 10-substituted 4-dedimethylamino minocycline derivative. In another embodiment, the 10-substituted tetracycline compound is a 10-substituted sancycline derivative. In another embodiment, the 10-substituted tetracycline compound is a 10-substituted 4-dedimethylamino sancycline derivative.

The invention also pertains to 10-substituted tetracycline compounds of formula VI:

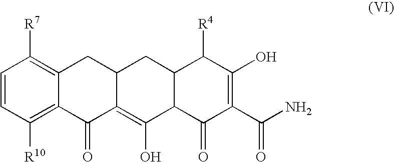

wherein:

R⁴ is amino or hydrogen;

R⁷ amino or hydrogen; and

R¹⁰ is hydrogen, substituted or unsubstituted C₁-C₅ alkyl, substituted or unsubstituted heterocyclyl, substituted or unsubstituted phenyl, substituted or unsubstituted heteroaryl, substituted or unsubstituted acyl, or substituted or unsubstituted imine;

or a pharmaceutically acceptable salt, ester or prodrug thereof.

In an embodiment, R⁴ is a dialkylamino group (e.g., dimethylamino). In another embodiment, R⁷ is a dialkylamino group (e.g., dimethylamino). In another embodiment, R⁴ and R⁷ are each dimethylamino.

In one embodiment, R¹⁰ is hydrogen.

In another embodiment, R¹⁰ is a substituted or unsubstituted heteroaryl group. In another embodiment, R¹⁰ is a substituted or unsubstituted phenyl group. The heteroaryl R¹⁰ group or the phenyl R¹⁰ group can be substituted with any substituent which allows the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In a further embodiment, the phenyl R¹⁰ group or the heteroaryl R¹⁰ group is substituted with substituted or unsubstituted alkyl. Examples of substituents of the alkyl include heterocycles such as, morpholine, piperidine, and pyrrolidine. In another further embodiment, the phenyl R¹⁰ group or the heteroaryl R¹⁰ group is substituted with an amino group. The amino group also may be further substituted e.g., with an alkyl, alkenyl, alkynyl, carbonyl, alkoxy or aryl (e.g., substituted or unsubstituted, heteroaryl, phenyl, etc.) group. The amino substituent may be substituted with any substituent or combination of substituents which allow it to perform its intended function. Examples of such substituents include halogens (e.g., fluorine, chlorine, bromine, iodine, etc.), amino (e.g., which can in turn be substituted with an alkyl, carbonyl, alkenyl, alkynyl, or aryl moiety), and arylamino (e.g., phenylamino).

The phenyl R¹⁰ group or the heteroaryl R¹⁰ group may also be substituted with alkoxy groups. Examples of alkoxy groups include, but are not limited to, methoxy, ethoxy, propoxy, butoxy, perfluoromethoxy, perchloromethoxy, methylenedioxy, etc. The phenyl group or the heteroaryl group may also be substituted with an amide group such as a carbamate moiety (e.g., an alkoxycarbonylamino group).

The heteroaryl R¹⁰ group also may be substituted or unsubstituted biaryl, e.g., naphthyl, fluorenyl, etc. The biaryl R¹⁰ group can be substituted with any substituent which allow it to perform its intended function. Examples of substituents include but are not limited to, alkyl, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In an embodiment, R¹⁰ is a heteroaryl group substituted with amino or formyl.

Examples of heteroaryl R¹⁰ moieties include, but are not limited to, furanyl, imidazolyl, benzothiophenyl, benzofuranyl, quinolinyl, isoquinolinyl, pyridinyl, pyrazolyl, benzodioxazolyl, benzoxazolyl, benzothiazolyl, benzoimidazolyl, thiofuranyl, oxadiazolyl, pyrrolyl, indolyl, thienyl, pyrimidyl, pyrazinyl, purinyl, pyrazolyl, oxazolyl, isooxazolyl, naphthridinyl, thiazolyl, isothiazolyl, and deazapurinyl. In certain embodiments, the heteroaryl R¹⁰ group is oxazolyl.

In another embodiment, R¹⁰ is substituted or unsubstituted alkyl. The alkyl group can be a straight or branched chain, e.g., methyl, ethyl, i-propyl, n-propyl, n-butyl, i-butyl, t-butyl, pentyl, hexyl. etc. The alkyl group may also comprise a ring, e.g., a cycloalkyl (e.g., cyclopentyl, cyclohexyl, cyclopropyl, or cyclobutyl). The alkyl R¹⁰ group may be substituted with any substituent or combination of substituents which allows the compound to perform its intended function. Examples of substituents include, but are not limited to, alkenyl, halogen, hydroxyl, alkoxy, alkylcarbonyloxy, alkyloxycarbonyl, carboxy, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

In certain embodiments, the alkyl group is substituted with an amino, hydroxy, carboxy, carbonyl (e.g., substituted carbonyl), heterocyclic or aryl groups. Examples of heterocyclic or aryl groups include, for example, furanyl, imidazolyl, benzothiophenyl, benzofuranyl, quinolinyl, isoquinolinyl, benzodioxazolyl, benzoxazolyl, benzothiazolyl, benzoimidazolyl, indolyl, thienyl, pyridinyl, pyrazolyl, pyrimidyl, pyrazinyl, purinyl, pyrazolyl, oxazolyl, isooxazolyl, naphthridinyl, thiazolyl, isothiazolyl, and deazapurinyl. In a further embodiment, the aryl group is pyridinyl.

In another embodiment, R¹⁰ is substituted or unsubstituted heterocyclyl. The heterocyclyl R¹⁰ group can be substituted with any substituent which allow the tetracycline compound to perform its intended function. Examples of substituents include, but are not limited to, alkyl, alkenyl, halogen, hydroxyl, alkoxy, alkylcarbonyloxy, alkyloxycarbonyl, arylcarbonyloxy, alkoxycarbonylamino, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, aminoalkyl, arylalkylcarbonyl, alkenylcarbonyl, alkoxycarbonyl, silyl, aminocarbonyl, alkylthiocarbonyl, phosphate, aralkyl, phosphonato, phosphinato, cyano, amino, acylamino, amido, imino, sulfhydryl, alkylthio, sulfate, arylthio, thiocarboxylate, alkylsulfinyl, sulfonato, sulfamoyl, sulfonamido, nitro, cyano, azido, heterocyclyl, alkylaryl, aryl and heteroaryl.

Examples of heterocyclyl R¹⁰ moieties include, but are not limited to, pyrrolidinyl, dioxolanyl, imidazolidinyl, pyrazolidinyl, piperidinyl, dioxanyl, morpholinyl, dithianyl, thiomorpholinyl, piperazinyl and trithianyl. In one embodiment, the heterocyclyl R¹⁰ group is piperidinyl. In another embodiments, the heterocyclyl R¹⁰ group is tetrahydropyran. In another embodiment, the heterocyclyl moieties are saturated. In another embodiment, the heterocyclyl moieties are partially unsaturated.

In another embodiment, R¹⁰ is substituted or unsubstituted acyl. The acyl R¹⁰ group can be substituted with any substituent which allow the tetracycline compound to perform its intended function. Examples of substituents include, but are not limited to, alkyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. In a further embodiment, R⁹ is acetyl.

In another embodiment, R¹⁰ is substituted or unsubstituted imine. The imine R¹⁰ group can be substituted with any substituent which allow the tetracycline compound to perform its intended function. Examples of substituents include, but are not limited to, alkyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The invention also pertains to the 10-substituted tetracycline compounds shown in Table 2, such as compounds Q and AY.

Also included are pharmaceutically acceptable salts, esters and prodrugs of the compounds of formulae VI and those shown in Table 2.

Table 2 includes several examples of tetracycline compounds.

TABLE 2

| | Com- |
|---|---|
| | pound |
| Structure | ID |
| 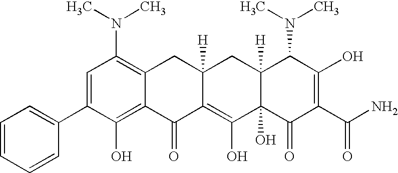 | A |
| 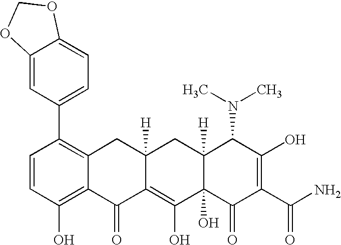 | B |
| 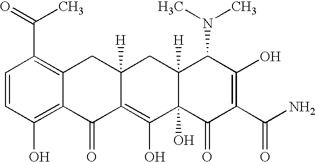 | C |
| 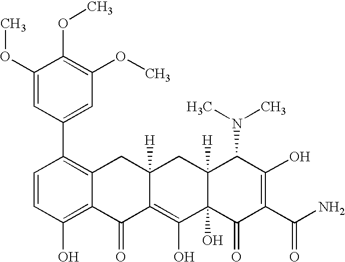 | D |
| 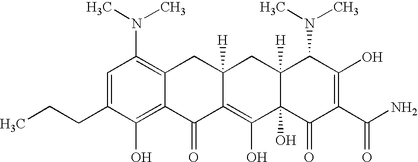 | E |
| 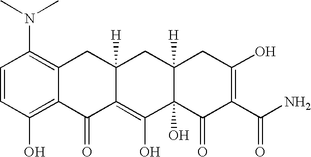 | F |
| 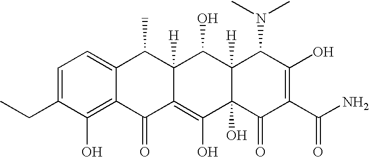 | G |
| 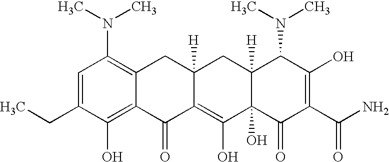 | H |
| 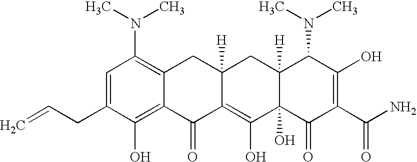 | I |
| 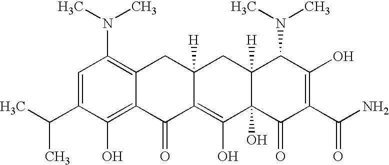 | J |
| 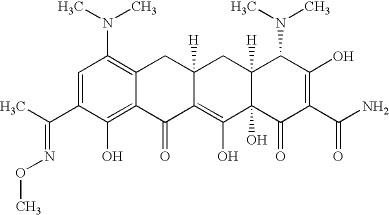 | K |
| 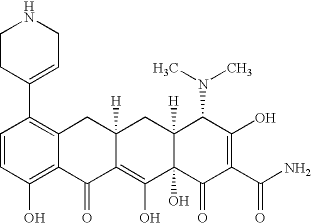 | L |
| 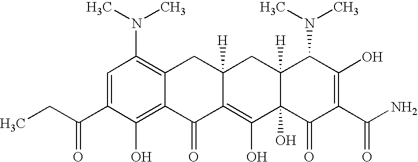 | M |
| 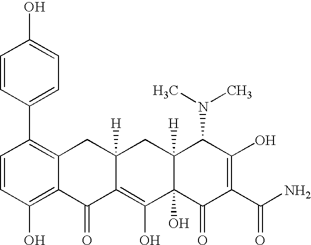 | N |
| 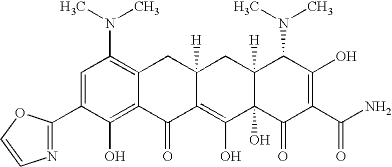 | O |
| 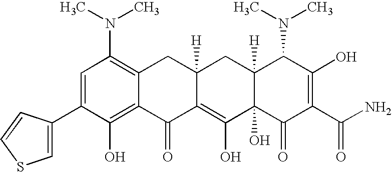 | P |
| 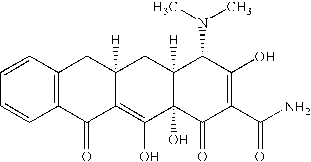 | Q |
| 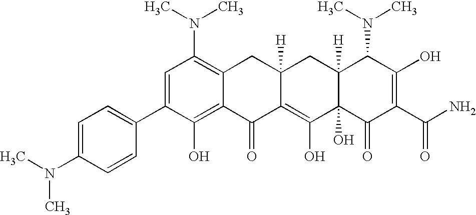 | R |
| 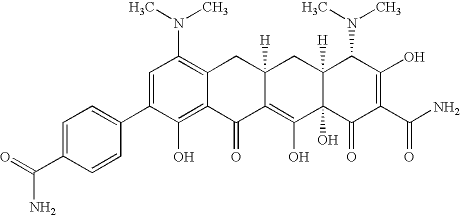 | S |
| 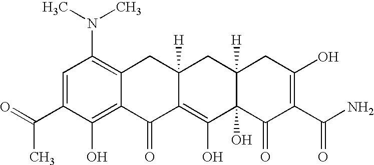 | T |
| 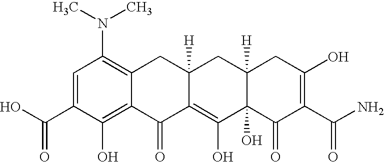 | U |
| 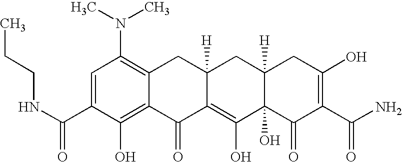 | V |
| 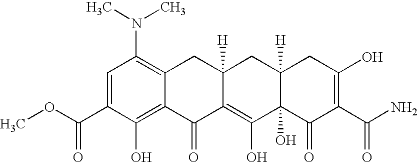 | W |
| 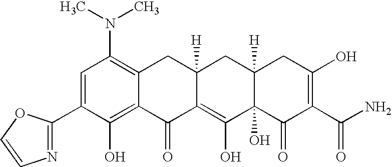 | X |
| 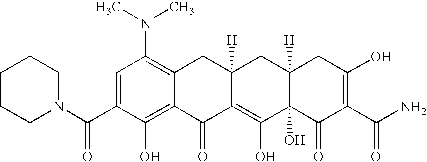 | Y |
| 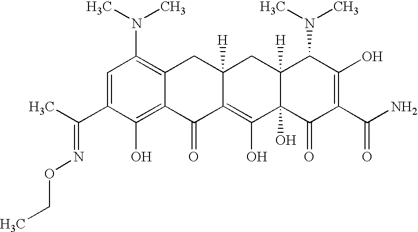 | Z |
| 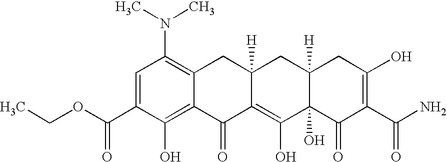 | AA |
| 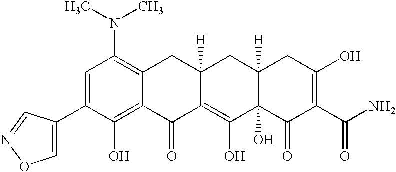 | AB |
| 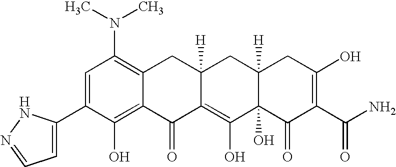 | AC |
| 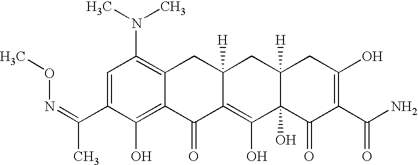 | AD |
| 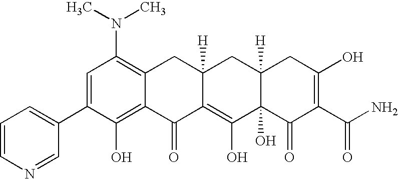 | AE |
| 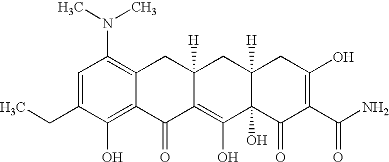 | AF |
| 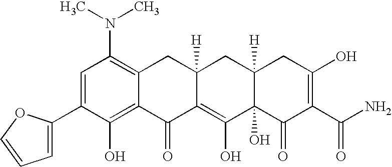 | AG |
| 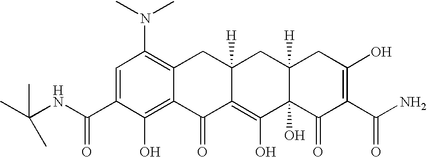 | AH |
| 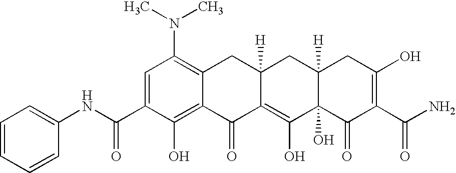 | AI |
| 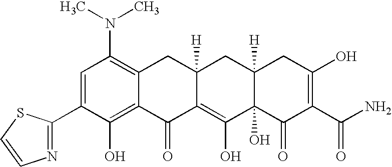 | AJ |
| 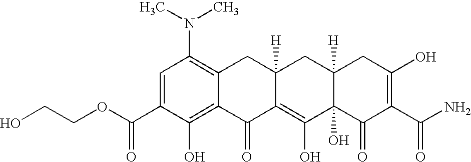 | AK |
| 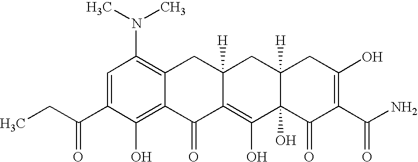 | AL |
| 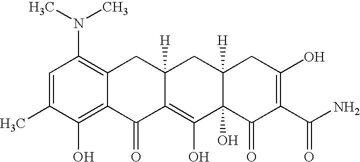 | AM |
| 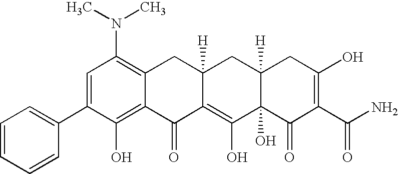 | AN |
| 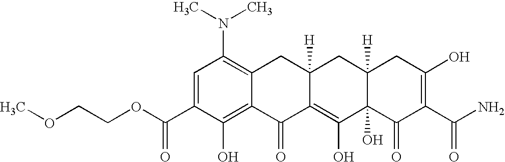 | AO |
| 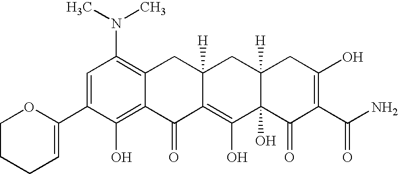 | AP |
| 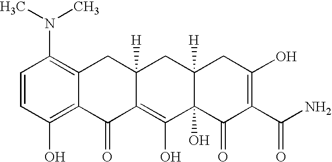 | AQ |
| 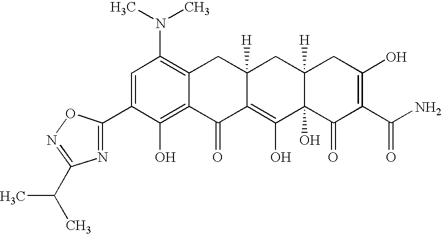 | AR |
| 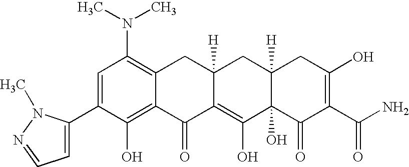 | AS |
| 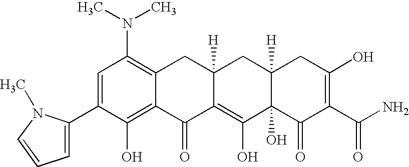 | AT |
| 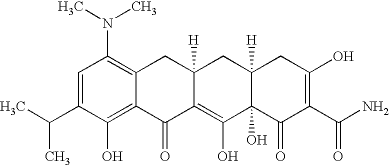 | AU |
| 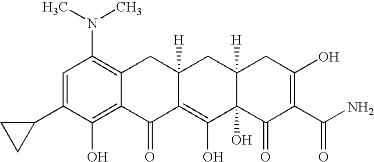 | AV |
| 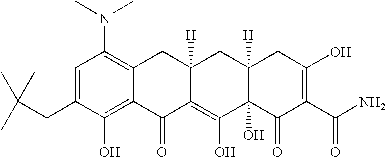 | AW |
| 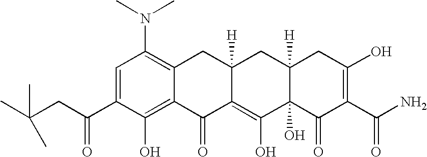 | AX |
| 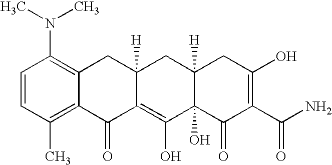 | AY |
| 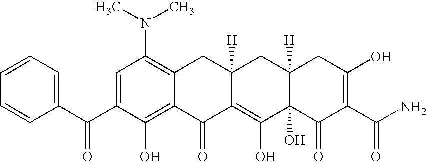 | AZ |
| 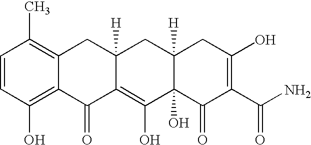 | BA |
| 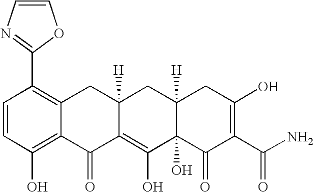 | BB |
| 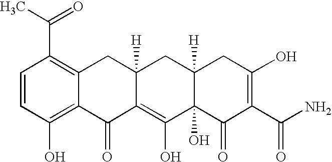 | BC |
| 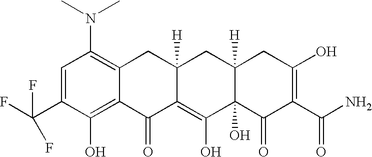 | BD |

### V. Synthetic Methods for the Synthesis of Tetracycline Compounds

The tetracycline compounds of the invention can be synthesized using the methods described in the Schemes and Examples below.

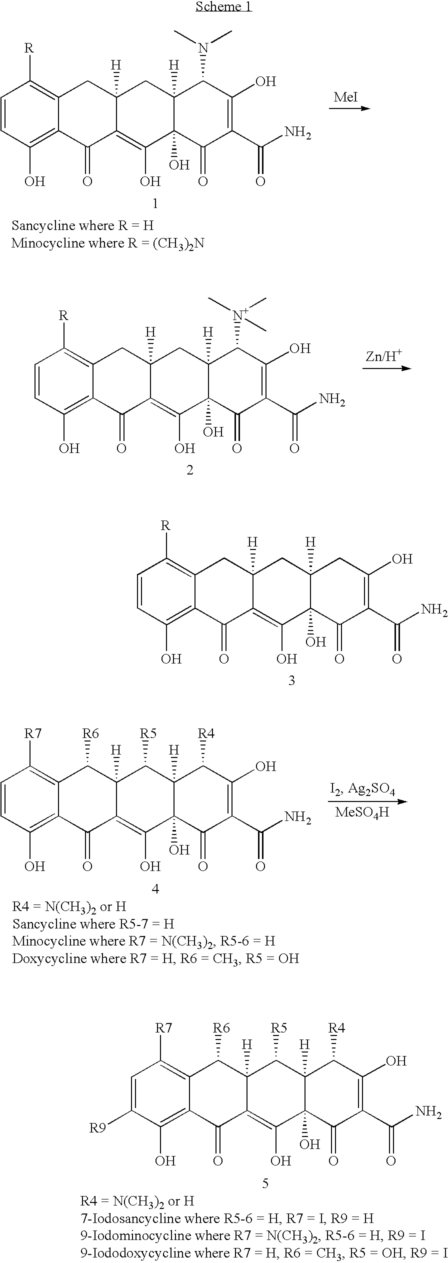

Synthesis of 4-trimethylammonium tetracyclines 2. The HCl salt of minocycline or sancycline (0.406 mol) was suspended in 3 L water. The pH was adjusted to 6.5-7.0 using NaHCO₃ (68 g, 0.812 mol for minocycline and 34 g, 0.406 mol for sancycline) in 3 portions. The solution was then extracted with 2×1.5 L CH₂Cl₂. The solution was concentrated to dryness to give the tetracycline as the free base 1. The free base was then dissolved in tetrahydrofuran (1.6 L) in a 3 L 3-necked flask equipped with an over-head stirrer and a temperature probe while under argon. Methyl iodide (289 g, 2.03 mol) was added and the solution was heated at 40-45° C. for approximately 16 hours; at which point it was verified that the reaction was complete via LCMS. The solution was then poured into 6 L of heptane while on an ice bath and stirred for at least 20 minutes at <5° C. The precipitate was filtered and washed with hexane (400 mL). The solid was dried under reduced pressure to a constant weight to give 220 g, 0.366 mol, methylammonium salt of minocycline or 190 g, 0.340 mol, of the methylammonium salt of sancycline.

Synthesis of 4-dedimethylamino minocycline or 4-dedimethylamino sancycline 3. In a 3 L, 3-necked round bottom flask equipped with an overhead stirrer, temperature probe, a mixture of 200 mL dimethylformamide (DMF), 50 mL trifluoroacetic acid (TFA), and 15 mL water was cooled on an ice bath to <5° C. 4-methylammonium minocycline or 4-methylammonium sancycline (0.166 mol) was then added. Zn powder (14 g, 100 mesh) was added in 6 portions approximately every 30 minutes (˜2.33 g each addition). The reaction was monitored by LCMS. When less than 10% of the tetracycline starting material was remaining, the solution was filtered through a bed of Celite® and was washed with 500 mL water. The solution was then poured into 2 L of water and the pH was adjusted with aqueous ammonia to 2.5. The aqueous solution was extracted first with 2×1 L dichloromethane. The combined organic layers were back-washed with 1 L water, dried on sodium sulfate, filtered and concentrated under reduced pressure to an oil, to give 0.100 mol of 4-dedimethylamino minocycline or 4-dedimethylamino sancycline.

Synthesis of iodotetracyclines 5. In a 2 L round bottom flask, 0.115 mol of tetracycline starting material 4 was dissolved in 350 mL methanesulfonic acid. Following, Ag₂SO₄ (75 g, 0.24 mol) and iodine (61.5 g, 0.24 mol) were added and the mixture was stirred for 3 hours. Upon completion of the reaction as determined by LCMS, the mixture was poured into 4% aqueous sodium sulfite (3.5 L) and was stirred for one hour. The solution was filtered through a bed of Celite® and washed with 200 ml of water. The aqueous layer was loaded onto a column containing divinylbenzyl resin. A gradient of 20-80% organic (1:1 methanol:acetonitrile) in water with an overall trifluoroacetic acid of 1.0% was used to elute product 5. The combined fractions were reduced of organic solvent using rotary evaporation, pH adjusted with aqueous NaHCO₃ to pH 7 and extracted with methylene chloride to give 0.95 mol of product 5 as the free base.

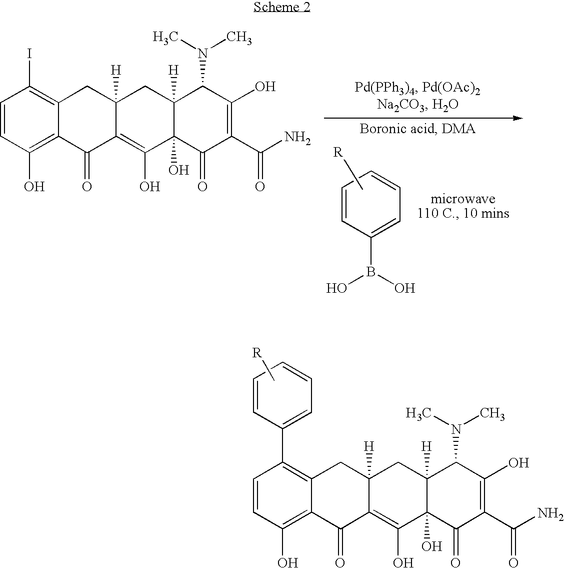

General Procedure for 7-phenyl tetracycline compounds (modified from Nelson, et al., JOC, 2003, 68 (15): 5838-5851). 7-iodosancycline (200 mg, 0.37 mmol) was combined with Pd(PPh₃)₄ and Pd(OAc)₂ (0.037 mmol each) in dimethylacetamide (15 mL) and degassed with argon (Ar). Separately, Na₂CO₃ (117 mg, 1.11 mmol in 5 mL of water) was purged with Ar for 10 min prior to addition by syringe into the reaction solution. This was followed by the addition of an Ar-degassed solution of phenylboronic acid (90 mg, 0.74 mmol in 5 mL of DMA). The reaction mixture was heated to and maintained at 110° C. using microwaves for 10 min. The solution was filtered through Celite® and the solvent removed in vacuo to produce the crude material. Final material was purified by preparative RP-HPLC.

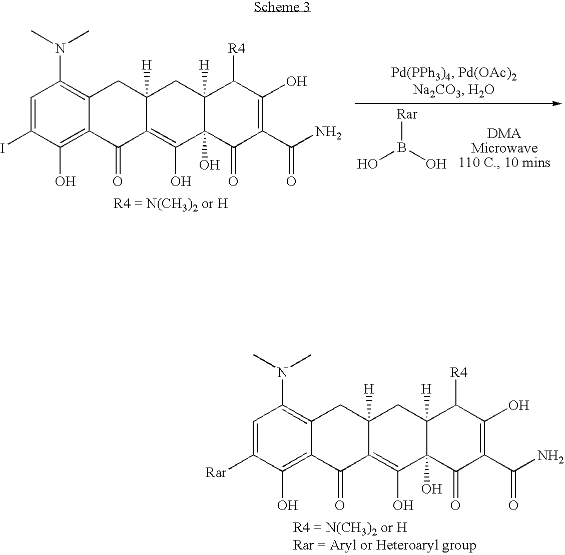

General Procedure for 9-phenyl tetracycline compounds (modified from Nelson, et al., JOC, 2003, 68 (15): 5838-5851). 9-iodotetracycline (0.37 mmol) was combined with Pd(PPh₃)₄ and Pd(OAc)₂ (0.037 mmol each) in dimethylacetamide (15 mL) and degassed with Ar. Separately, Na₂CO₃ (117 mg, 1.11 mmol in 5 mL of water) was purged with Ar for 10 min prior to addition by syringe into the reaction solution. This was followed by the addition of an Ar-degassed solution of phenylboronic acid (90 mg, 0.74 mmol in 5 mL of DMA). The reaction mixture was heated in microwave at 110° C. for 10 min, monitored via HPLC. The solution was filtered through Celite® and the solvent removed in vacuo to produce the crude material. Final material was purified by preparative RP-HPLC.

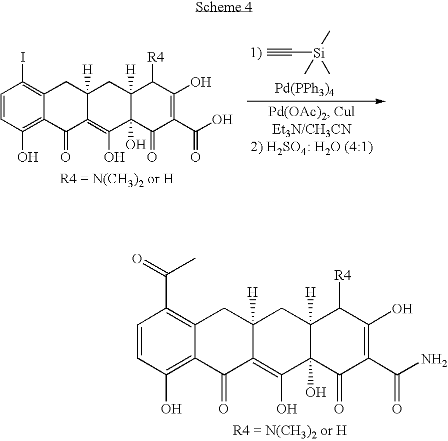

General Procedure for tetracycline alkyne derivatives (modified from Nelson, et al., JOC, 2003, 68 (15): 5838-5851). A 1-mmol sample of 7-iodotetracycline, 50 mg of tetrakistriphenylphosphine palladium(0) catalyst or equivalent, 12 mg of Pd(OAc)₂, and 32 mg of CuI were dissolved in 10 mL of acetonitrile. Triethylamine (2-5 mL) and 3-5 mmol of alkyne were added and the mixture was vigorously stirred between room temperature and 70° C. for 2-24 h. Filtration through Celite® and removal of the solvent in vacuo produced crude 7-alkyne. The alkyne was converted to the acetyl by dissolving the crude material in H₂SO₄:H₂O (4:1) and stirring at room temperature for 2-4 hours. The final product was purified by preparative RP-HPLC.

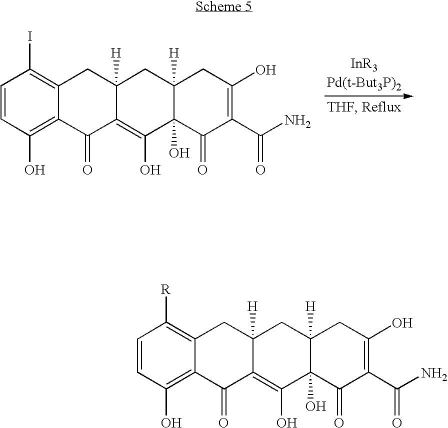

General Procedure for 7-alkylsancyclines. A 1000 mL 2- or 3-neck round-bottomed flask with reflux condenser was charged with anhydrous InCl₃ (12.1 g, 40.5 mmol) and dried under vacuum with a heat gun. After flask was cooled to ambient temperature and flushed with argon, anhydrous tetrahydrofuran (THF) (240 mL) was added. The solution was cooled to −78° C. and RMgBr(Cl) (122 mmol) as solution in THF was added. After 15 minutes, the solution was allowed to slowly warm to room temperature to form a clear heterogeneous solution. To the reaction flask was added 7-iodosancycline or 7-iodo-4-dedimethylaminosancycline (36 mmol) and Pd(t-Bu₃P)₂ (0.920 g, 1.80 mmol). The solution was heated to reflux under argon until complete (approximately 1-8 h). After cooling to ambient temperature, the solution was quenched with MeOH (1 mL) and poured into a stirring cold solution of 1M HCl (3 L). After 1 h, the solution was filtered through a pad of Celite® rinsing with water. The water solution was loaded onto a large fitted funnel containing a bed of prepared divinylbenzene (DVB) resin. At first, cold water (500 mL) was eluted then a gradient of cold acetonitrile/water was eluted in (500 mL) fractions. The fractions containing product were concentrated under reduced pressure and then dried under high vacuum overnight to afford 10 g in 57% yield. The fractions can be purified further by preparatory RP-HPLC.

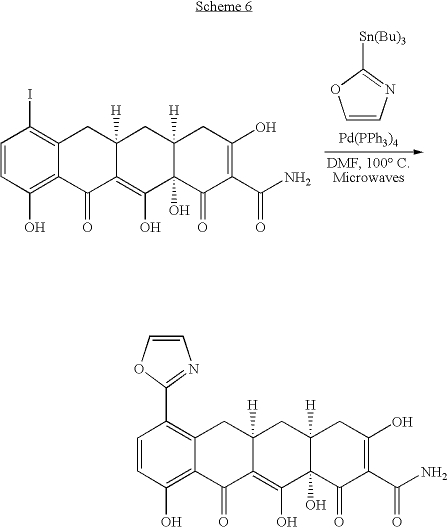

Synthesis of 7-(2-oxazolyl)-4-dedimethylamino sancycline. To a 20 mL Biotage® microwave vial was added a solution of anhydrous 7-iodo-4-dedimethylamino sancycline freebase (3.5 mmol), 2-oxazolylstannane (4.38 mmol), Pd(PPh₃)₄ (0.35 mmol) in DMF (20 mL). The secured vial was placed into a Biotage® microwave reactor with a temperature setting of 100° C. for 10 min. The reaction was poured into a solution of 1% TFA/H₂O (150 mL). The solution was filtered through a plug of Celite® rinsing with 1% TFA water solution. The solution was loaded onto a previously prepared funnel of DVB resin (3×10 cm packed DVB column). After loading, water (100 mL) was eluted and finally CH₃CN to elute the desired product. The yellow solution was concentrated under reduced pressure and further purified by preparatory RP-HPLC.

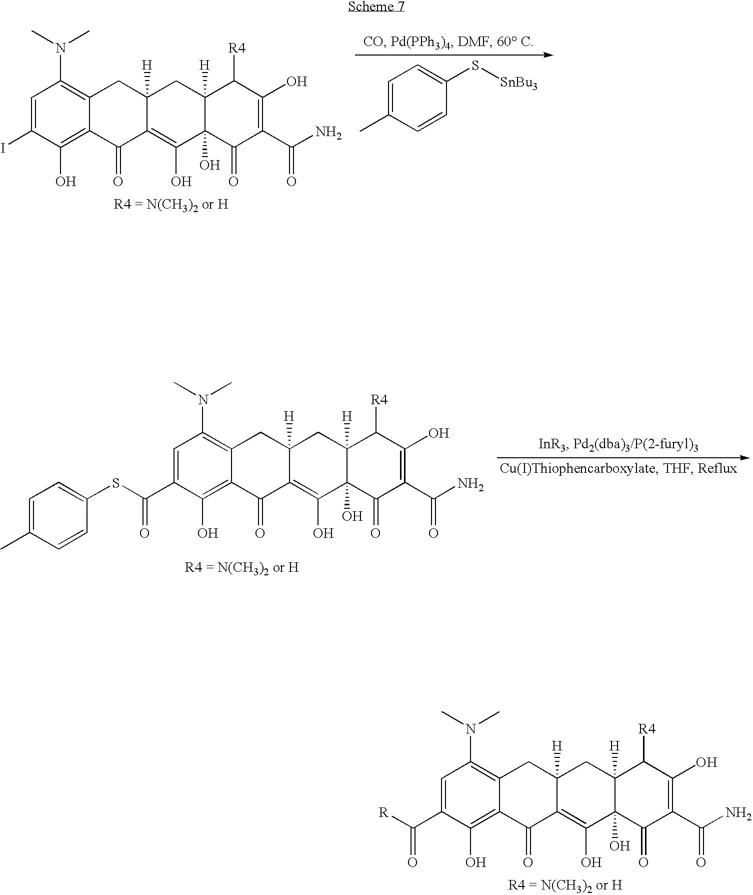

General Procedure for 9-(4-methylphenyl)thiocarboxylacyl minocyclines. To a solution of anhydrous 9-iodominocycline or 9-iodo-4-dedimethylaminominocycline freebase (35.0 mmol), 4-methylphenylthiotributyltin (15.9 g, 38.5 mmol) and Pd(PPh₃)₄ (2.02 g, 1.75 mmol) in anhydrous DMF (175 mL) was bubbled carbon monoxide (CO) for 15 min, then heated to 60° C. with a large balloon filled with CO affixed to the flask to maintain a positive pressure of CO. After 12 h, the reaction was cooled to room temperature, poured into a cold 1:1 solution of 1% TFA/H₂O (500 mL) and methyl tert-butyl ether (MTBE) (500 mL). After separating layers, the organic layer was back extracted with 1% TFA/H₂O (500 mL). The combined water layers were loaded onto a previously prepared funnel of DVB resin (7×15 cm packed DVB column). After loading, a cold solution of 1M NaOAc was eluted until the eluent became basic (approximately 300 mL), then water (400 mL) and finally 1:1 CH₃CN/THF to elute the desired product. The yellow solution was concentrated under reduced pressure and further dried under high vacuum overnight to afford 18.5 g as an orange solid in 87% yield.

Triorganoindium Procedure for 9-alkylacyl minocyclines. To a solution of 9-(4-methylphenyl)thiocarboxylacyl minocycline or 9-(4-methylphenyl)thiocarboxylacyl-4-dedimethylamino minocycline (2.80 mmol), copper(I) thiophene-2-carboxylate (CuTC) (0.801 g, 4.20 mmol), tris(dibenzylideneacetone) dipalladium(0) (Pd₂(dba)₃) (0.064 g, 0.070 mmol) and P(2-furyl)₃ (0.130 g, 0.560 mmol) in anhydrous THF (5 mL) under argon was added a 0.1M solution of previously prepared R₃In (56.0 mL, 5.60 mmol), then the solution was heated to reflux until reaction was complete (4-12 h). After cooling to room temperature, the solution was poured into cold 0.1M HCl (mL) and stirred for 1 h. To the solution was added Celite® and then filtered through a large plug of Celite® rinsing with cold water. The cold solution was loaded onto a prepared column of DVB resin (3×10 cm packed DVB column). When the loading was complete, water (300 mL) was eluted, and then CH₃CN was eluted until the eluent became colorless. The yellow solution was concentrated under reduced pressure, then further purified by preparatory RP-HPLC.

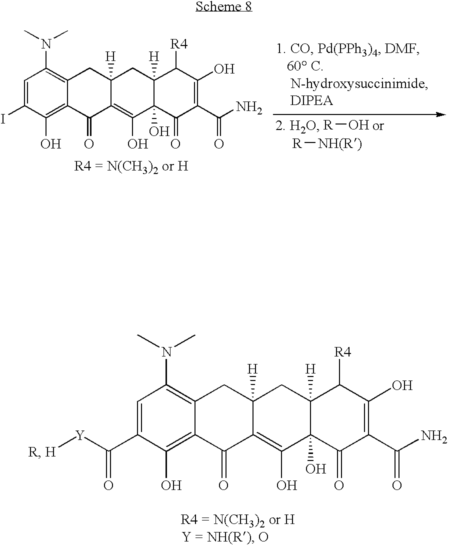

General Procedure for 9-acylminocyclines. To a 500 mL flask was added (4.30 mmol) 4-dedimethylamino-9-iodo minocycline or 9-iodo minocycline free base, N-methyl-2-pyrrolidone (NMP) (37 mL), and N-hydroxysuccinimide (3.9 g, 38 mmol). To remove residual water from the above reactants toluene was added (37 mL). The flask was then placed on the rotary evaporator (5 mm Hg, 45° C.) until all the toluene was evaporated. The flask was backfilled with argon and the contents were then transferred via cannula to a dry 500 L flask. To the 0.5 L flask was added tetrakis(triphenylphosphine)palladium(0) (2.00 g, 1.67 mmol) and diisopropylethylamine (DIEA) (2.60 mL, 1.48 mmol). The flask was placed under vacuum (20 mm Hg) and purged 3× with carbon monoxide. The flask was then heated to 60° C. under 1.0 atm of carbon monoxide and let stir for 1 h until all starting material was consumed and a peak for the corresponding NHS-ester intermediate was formed as determined via LCMS. Subsequently, the corresponding amine, alcohol or water (438 mmol) and DIEA (4.0 mL, 38 mmol) was added and the reaction was heated in a microwave reactor for 1 min at 100° C. The reaction was added to acetonitrile (150 mL) followed by water (0.8 L) and the pH was lowered to 2 using trifluoroacetic acid. The solution was then filtered through Celite® to remove the catalyst, loaded onto a reverse phase column and the crude product was purified by HPLC (C18, linear gradient 30-45% acetonitrile in water with 0.2% formic acid).

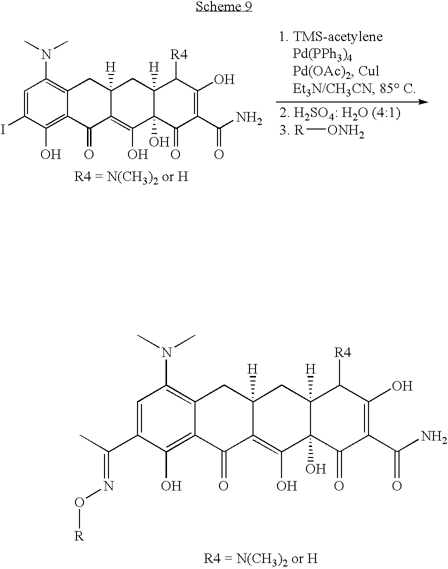

General Procedure for 9-ethoxyimino-ethyl minocyclines. In a 100 mL, 3-necked flask, 9-iodo-4-dedimethylamino minocycline or 9-iodo minocycline (6.11 mmol), palladium (II) acetate (0.071 g, 0.31 mmol), CuI (0.123 g, 0.611 mmol), [Pd(PPh₃)₄] (0.363 g, 0.31 mmol), and a stir bar were charged. Acetonitrile (30 mL) was added and the reaction flask was purged with Ar for 1 min. The trimethylsilylacetylene (1.8 mL, excess) was added to the reaction mixture followed by the addition of Et₃N (3.4 mL). The reaction flask was heated to 85° C. (bath temp) and allowed to stir. A reaction aliquot taken after 5 min, showed the completion of the reaction by LCMS [(ESI+) m/z Theor. Calc. 510.62, Obs. 511.71 (MH⁺)]. The reaction mixture was filtered hot through a bed of Celite®, and the filter bed washed with 3×10 mL of MeCN. The combined filtrate was first evaporated to dryness and further dried under high vacuum for 12 h. To the flask containing the dried product, was added an 80% aqueous TFA solution (40 mL) and stirred at room temperature for 5 minutes, followed by stirring at 80° C. for 5 min. At this stage the reaction sample contained 2 components—the terminal acetylene (MS: obs. m/z=439) and the desired product (MS: obs m/z=457.20). An 80% solution of H₂SO₄ was added (while hot) to the reaction mixture over approximately 60 seconds. The LCMS confirmed the complete consumption of the starting material and formation of the desired product. The reaction mixture was poured over ice, the resulting solution/suspension was filtered over Celite®, and the black precipitate was washed with 3×50 mL of water. The filtrate was cooled down to 4-6° C. by adding approximately 300 g of ice. The cold aqueous solution was then neutralized by adding solid NaHCO₃ (approximately 110 g) in small portions until the pH of the solution/suspension was approximately 5. The suspension was extracted in 2×300 mL portions of CH₂Cl₂, the organic extract was dried over anhydrous Na₂SO₄ and evaporated to dryness first under rotary evaporator and then under high vacuum. The material was dissolved in methanol and treated with appropriate alkoxyamine and allowed to stir for 3 h. The reaction was monitored with LCMS, and upon completion of the reaction, the crude product was purified by preparative column chromatography (C18, linear gradient 15-55% acetonitrile in 20 mM aqueous triethanolamine and TFA, pH 7.4).

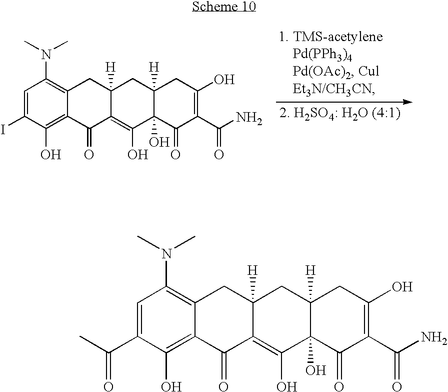

Synthesis of 9-acetyl-4-dedimethylamino minocycline. In a 100 mL, 3-necked flask, 9-iodo-4-dedimethylamino minocycline (4.001 g, 6.11 mmol), palladium (II) acetate (0.071 g, 0.31 mmol), CuI (0.123 g, 0.611 mmol), [Pd(PPh₃)₄] (0.363 g, 0.31 mmol), and a stir bar were charged. Acetonitrile (30 mL) was added and the reaction flask was purged with Ar for 1 min. The trimethylsilylacetylene (1.8 mL, excess) was added to the reaction mixture followed by the addition of Et₃N (3.4 mL). The reaction flask was heated to 85° C. (bath temp) and allowed to stir. A reaction aliquot taken after 5 min, showed the completion of the reaction by LCMS [(ESI+) m/z Theor. Calc. 510.62, Obs. 511.71 (MH⁺)]. The reaction mixture was filtered hot through a bed of Celite®, and the filter bed washed with 3×10 mL of MeCN. The combined filtrate was first evaporated to dryness and further dried under high vacuum for 12 h. To the flask containing the dried product, was added an 80% aqueous TFA solution (40 mL) and stirred at room temperature for 5 minutes, followed by stirring at 80° C. for 5 min. At this stage the reaction sample contained 2 components—the terminal acetylene (MS: obs. m/z=439) and the desired product (MS: obs m/z=457.20). An 80% solution of H₂SO₄ was added (while hot) to the reaction mixture over approximately 60 seconds. The LCMS confirmed the complete consumption of the starting material and formation of the desired product. The reaction mixture was poured over ice, the resulting solution/suspension was filtered over Celite®, and the black precipitate was washed with 3×50 mL of water. The filtrate was cooled down to 4-6° C. by adding approximately 300 g of ice. The cold aqueous solution was then neutralized by adding solid NaHCO₃ (approximately 110 g) in small portions until the pH of the solution/suspension was approximately 5. The suspension was extracted in 2×300 mL portions of CH₂Cl₂, the organic extract was dried over anhyd. Na₂SO₄ and evaporated to dryness, first under rotary evaporator and then under high vacuum. The crude product was purified by preparative chromatography (C18, linear gradient 15-40% acetonitrile in water with 0.1% TFA, 280 nm).

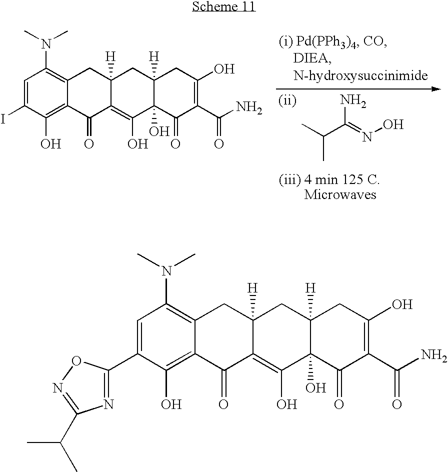

Synthesis of 9-(3-isopropyl-1,2,4-oxadiazoyl)-4-dedimethylamino minocycline. To a 500 mL flask was added (4.00 g, 8.60 mmol) 4-dedimethylamino-9-iodo minocycline free base, NMP (50 mL), N-hydroxysuccinimide (3.9 g, 38 mmol), a stir bar, tetrakis(triphenylphosphine)palladium(0) (2.00 g, 1.67 mmol) and DIEA (3.0 mL, 1.7 mmol). The flask was placed under vacuum (20 mm Hg) and purged 3× with carbon monoxide. The flask was then heated to 60° C. under 1.0 atm of carbon monoxide and stirred for 1 h until all 4-dedimethlyamino-9-iodo minocycline was consumed and a peak for the corresponding 9-NHS-ester 4-dedimethylamino minocycline intermediate (M+1) of 556 M/Z was formed as determined via LCMS. The NHS-ester intermediate was then reacted with N′-hydroxy-2-methylpropanimidamide (2.0 g, 19.6 mmol) at room temperature for 2 h, to give the noncyclized intermediate (M+1) of 543 M/Z as determined via LCMS. The noncyclized intermediate was isolated by adding it to 50 mL acetonitrile followed by dilution of the reaction mixture with water to a total volume of 2.0 L. The water was adjusted to a pH of 2.0 using trifluoroacetic acid. The aqueous solution was then filtered and loaded onto a plug of DVB resin and purified (10-60% MeCN, 0.1% TFA) to give 1 g of crude noncyclized intermediate. To noncyclized-intermediate (2.0 g, 3.7 mmol) in a 500 mL round bottom flask was added NMP (80 mL) and toluene (80 mL). To prevent hydrolysis during the subsequent cyclization step, residual water was removed from the noncyclized intermediate by subjecting it to rotary evaporation (5 mm Hg, 45° C.) until all the toluene/water was evaporated. The flask was backfilled with argon and diisopropylamine (2 mL, 1.13 mmol) was added. To facilitate cyclization, the contents were heated to 125° C. for 8 minutes using microwaves. The contents were then added to acetonitrile, diluted with water to a final volume of two liters and trifluoroacetic acid was added to a final pH of 2. The solution was then filtered through Celite® to remove the catalyst, loaded onto a reverse phase column and the crude product was purified by HPLC (C18, linear gradient 30-40% acetonitrile in water with 0.1% TFA). The fractions containing the final product were loaded onto a DVB plug, washed with aqueous HCl (1.0 L, 0.01 N) and eluted with methanol to give the HCl salt of 9-(3-isopropyl-1,2,4-oxadiazoyl)-4-dedimethylamino minocycline (280 mg, 0.53 mmol, 12%).

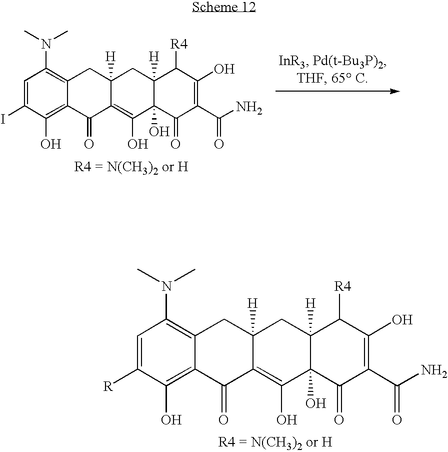

General Procedure for 9-Alkyl minocyclines. A 1000 mL 2 or 3 neck round-bottomed flask with reflux condenser was charged with anhydrous InCl₃ (12.1 g, 40.5 mmol) and dried under vacuum with a heat gun. After the flask was cooled to ambient temperature and flushed with argon, anhydrous THF (240 mL) was added. The solution was cooled to −78° C. and RMgBr(Cl) (122 mmol) as a solution in THF was added. After 15 min., the solution was allowed to slowly warm to room temperature to form a clear heterogeneous solution. To the reaction flask was added 9-iodo minocycline or 9-iodo-4-dedimethylamino minocycline (36 mmol) and Pd(t-Bu₃P)₂ (0.920 g, 1.80 mmol). The solution was heated to reflux under argon until complete (approximately 1-8 h). After cooling to ambient temperature, the solution was quenched with MeOH (1 mL) and poured into a stirring cold solution of 1M HCl (3 L). After 1 h, the solution was filtered through a pad of Celite® rinsing with water. The water solution was loaded onto a large fitted funnel containing a bed of prepared DVB resin. At first, cold water (500 mL) was eluted then a gradient of cold acetonitrile/water was eluted in (500 mL) fractions. The fractions containing product were concentrated under reduced pressure and then dried under high vacuum. Crude material was purified further by preparatory RP-HPLC.

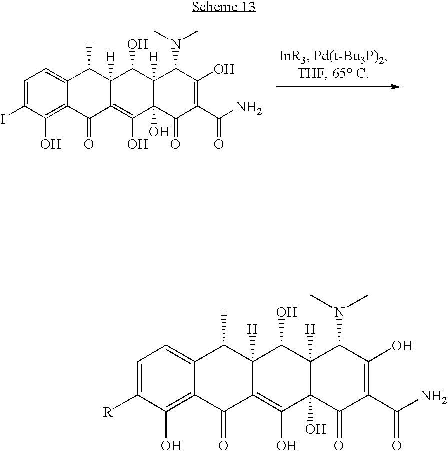

Synthesis of 9-ethyl doxycycline. A 1000 mL 2 or 3 neck round-bottomed flask with reflux condenser was charged with anhydrous InCl₃ (12.1 g, 40.5 mmol) and dried under vacuum with a heat gun. The flask was then cooled to ambient temperature, flushed with argon and anhydrous THF (240 mL) was added. The solution was cooled to −78° C. and EtMgBr (122 mmol) as solution in THF was added. After 15 min., the solution was allowed to slowly warm to room temperature to form a clear heterogeneous solution. To the reaction flask was added 9-iodo doxycycline (36 mmol) and Pd(t-Bu₃P)₂ (0.920 g, 1.80 mmol). The solution was heated to reflux under argon until complete (approximately 1-8 h). After cooling to ambient temperature, the solution was quenched with MeOH (1 mL) and poured into a stirring cold solution of 1M HCl (3 L). After 1 h, the solution was filtered through a pad of Celite® and rinsed with water. The water solution was loaded onto a large fitted funnel containing a bed of prepared DVB resin. At first, cold water (500 mL) was eluted. Then, a gradient of cold acetonitrile/water was eluted in (500 mL) fractions. Crude material was purified further by preparatory RP-HPLC.

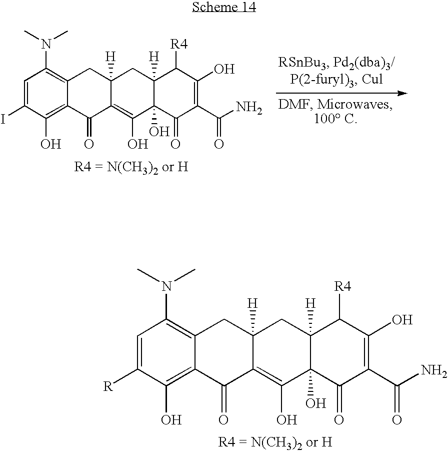

General Procedure for 9-substituted minocyclines through Stille coupling. To a solution of anhydrous 9-iodo minocycline or 9-iodo-4-dedimethylamino minocycline freebase (3.5 mmol), stannane (4.38 mmol), CuI (0.067 g, 0.350 mmol), P(2-furyl)₃ (0.163 g, 0.700 mmol) and Pd₂(dba)₃ (0.081 g, 0.088 mmol) in DMF (20 mL) were added in a 20 mL Biotage® microwave vial. The secured vial was placed into a Biotage® microwave reactor with a temperature setting of 100° C. for 10 min. The reaction was poured into a solution of 1% TFA/H₂O (150 mL). The solution was filtered through a plug of Celite® and rinsed with 1% TFA water solution. The solution was loaded onto a previously prepared funnel of DVB resin (3×10 cm packed DVB column). After loading the crude material, water (100 mL) was eluted and finally CH₃CN to elute the desired product. The yellow solution was concentrated under reduced pressure and further purified by preparatory RP-HPLC.

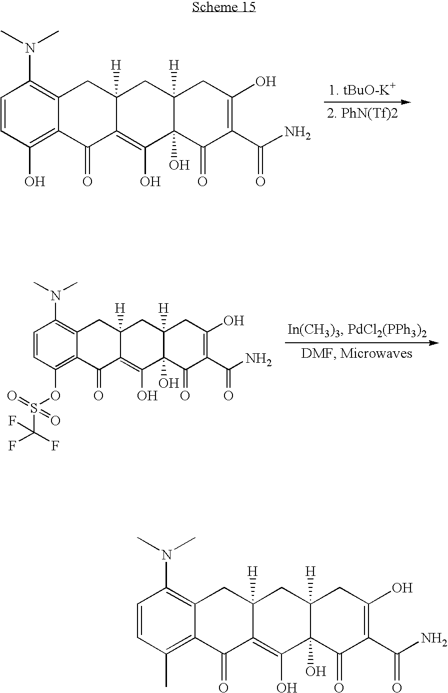

Synthesis of 10-methyl-4-dedimethylamino minocycline. To a solution of anhydrous freebase 4-dedimethylamino minocycline (25.0 mmol) in anhydrous THF under argon (163 mL) at 0° C. was added a 1M solution of potassium tert-butoxide (87.5 mL, 87.5 mmol) dropwise. After 45 min., N-phenylbis(trifluoromethanesulfonimide) (18.8 g, 52.5 mmol) was added at once. After 1 h, the solution was allowed to slowly warm to room temperature. After another 2 h, the solution was slowly poured into a vigorously stirring solution of 0.1M HCl and Celite®. After 15 min., the solution was filtered through a large plug of Celite® rinsing with 0.1M HCl. The water layer was loaded onto a DVB resin for purification. After the solution was loaded, a 0.1 M HCl solution was eluted, then CH₃CN with 1 mL conc. HCl was eluted where the yellow eluent was collected until it became colorless. The solution was concentrated under reduced pressure and further dried under high vacuum to afford 10-triflate intermediate. To a 200 mL round bottom flask was added THF (40 mL), a stir bar and InCl₃ (4.4 g, 20.0 mmol). The flask was then cooled to −78° C. by placing it in a dry ice bath. A solution of methylmagnesiumchloride in THF (20 mL, 3.0 N, 60 mmol) was slowly added to the stirred reaction over 5 minutes to generate a trimethyl-indium intermediate stock solution. The reaction was allowed to warm to room temperature. To the 10-triflate intermediate (0.61 mmol) was added N-methylpyrrolidone (10 mL), trans-dichlorobis(triphenylphosphine)palladium(II) (PdCl₂(PPh₃)₂) (1.0 g, 1.4 mmol) and the above trimethyl-indium intermediate stock solution (15 mL). The reaction was subject to microwave irradiation for a duration of 4 minutes at a temperature of 110° C. The reaction was then added to an aqueous solution (2.0 L) containing acetonitrile (10%) and TFA was added until a pH of 2 was reached. The solution was then filtered through Celite® to remove the catalyst, loaded onto a reverse phase column and purified by RP-HPLC.

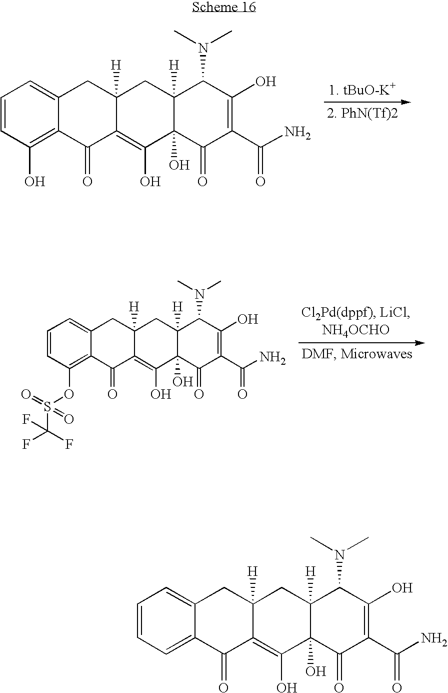

Synthesis of 10-deoxy sancycline. To a solution of anhydrous freebase sancycline (25.0 mmol) in anhydrous THF under argon (163 mL) at 0° C. was added a 1M solution of potassium tert-butoxide (87.5 mL, 87.5 mmol) dropwise. After 45 min, N-phenylbis(trifluoromethanesulfonimide) (18.8 g, 52.5 mmol) was added at once. After 1 h, the solution was allowed to slowly warm to room temperature. After another 2 h, the solution was slowly poured into a vigorously stirred solution of 0.1 M HCl and Celite®. After 15 min., the solution was filtered through a large plug of Celite® and rinsed with 0.1M HCl. The water layer was loaded onto a DVB resin for purification. After the solution was loaded, a 0.1 M HCl solution was eluted, then CH₃CN with 1 mL conc. HCl was eluted where the yellow eluent was collected until it became colorless. The solution was concentrated under reduced pressure and further dried via high vacuum to afford 10-triflate intermediate. To a solution of sancycline-10-triflate freebase (3.50 mmol) in DMF (10 mL) and H₂O (10 mL) was added ammonium formate (0.662 g, 10.5 mmol), LiCl (0.297 g, 7.00 mmol) and Cl₂Pd(dppf) (0.022 g, 0.175 mmol) in a 20 mL Biotage® microwave vial. The secured vial was placed into a Biotage® microwave reactor with a temperature setting of 100° C. for 7 min. After cooling, the vial was opened and poured into a 1% TFA/water solution. The solution was filtered through a plug of Celite® and rinsed with 1% TFA/water until the filtrate became colorless. The water solution was loaded onto a prepared DVB resin for semi-purification. After the solution was loaded, distilled water was eluted to remove salts, and then CH₃CN was eluted where the yellow eluent was collected until the eluent became colorless. The solution was concentrated under reduced pressure and further purified on preparatory chromatography on a reverse phase column. The combined fractions were concentrated under reduced pressure to afford a pale yellow solid.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

“Substituted alkyls” refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl(benzyl)). The term “alkyl” also includes the side chains of natural and unnatural amino acids.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

“Substituted alkenyls” refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

“Substituted alkynyls” refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino; diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties which contain the acyl radical (CH₃CO—) or a carbonyl group. It includes substituted acyl moieties. The term “substituted acyl” includes a carbonyl group (e.g., formyl or acetyl) where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “imine” includes compounds with a —C═N— group, e.g., an oxime group (—C═N—O—).

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term includes “alkyl amino” which comprises groups and compounds wherein the nitrogen is bound to at least one additional alkyl group. The term also includes “dialkyl amino” wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino, group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

The term “carbonyl” or “carboxy” includes compounds and moieties which contain a carbon connected with a double bond to an oxygen atom. The carbonyl can be further substituted with any moiety which allows the compounds of the invention to perform its intended function. For example, carbonyl moieties may be substituted with alkyls, alkenyls, alkynyls, aryls, alkoxy, aminos, etc. Examples of moieties which contain a carbonyl include aldehydes, ketones, carboxylic acids, amides, esters, anhydrides, etc.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, arylalkylaminocarbonyl, alkenylaminocarbonyl, alkylcarbonyl, arylcarbonyl, arylalkyl carbonyl, alkenylcarbonyl, aminocarbonyl, alkylthiocarbonyl, alkoxyl, phosphate, phosphonato, phosphinato, cyano, amido, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

The term “prodrug moiety” includes moieties which can be metabolized in vivo to a hydroxyl group and moieties which may advantageously remain esterified in vivo. Preferably, the prodrugs moieties are metabolized in vivo by esterases or by other mechanisms to hydroxyl groups or other advantageous groups. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters.

It will be noted that the structure of some of the tetracycline compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof.

### VI. Methods for Treating Rheumatoid Arthritis

The invention also pertains to methods for treating rheumatoid arthritis in subjects, by administering to a subject an effective amount of a tetracycline compound of the invention (e.g., of Formula I, II-A, II-B, III, IV-A, IV-B, V, VI or Table 2), such that the rheumatoid arthritis is treated.

The invention also pertains to methods for preventing rheumatoid arthritis in subjects, by administering to a subject an effective amount of a tetracycline compound of the invention (e.g., of Formula I, II-A, II-B, III, IV-A, IV-B, V, VI or Table 2), such that the rheumatoid arthritis is prevented.

The term “treating” includes curing as well as ameliorating at least one symptom of the state, disease or disorder, e.g., rheumatoid arthritis. The term “treating” does not include prophylaxis or prevention of a state, disease or disorder.

In another embodiment, the tetracycline compounds of the invention are substantially non-antibacterial. For example, non-antibacterial tetracycline compounds of the invention may have MIC values greater than about 4 μg/ml (as measured by assays known in the art and/or the assay given in Example 3).

Without being bound by theory, the efficacy of minocycline in rheumatoid arthritis is postulated to be linked to its immunomodulatory characteristics via inhibition of metalloproteinases and suppression of macrophage and T cell activation.

The present invention is related to inflammatory process associated states (IPAS). The term “inflammatory process associated state” includes states in which inflammation or inflammatory factors (e.g., matrix metalloproteinases (MMPs), nitric oxide (NO), TNF, interleukins, plasma proteins, cellular defense systems, cytokines, lipid metabolites, proteases, toxic radicals, adhesion molecules, etc.) are involved or are present in an area in aberrant amounts, e.g., in amounts which may be advantageous to alter, e.g., to benefit the subject. The inflammatory process is the response of living tissue to damage. The cause of inflammation may be due to physical damage, chemical substances, micro-organisms, tissue necrosis, cancer or other agents. Acute inflammation is short-lasting, lasting only a few days. If it is longer lasting however, then it may be referred to as chronic inflammation.

IPAS's include inflammatory disorders. Inflammatory disorders are generally characterized by heat, redness, swelling, pain and loss of function. Examples of causes of inflammatory disorders include, but are not limited to, microbial infections (e.g., bacterial and fungal infections), physical agents (e.g., burns, radiation, and trauma), chemical agents (e.g., toxins and caustic substances), tissue necrosis and various types of immunologic reactions.

Examples of inflammatory disorders include, but are not limited to, osteoarthritis, rheumatoid arthritis, acute and chronic infections (bacterial and fungal, including diphtheria and pertussis); acute and chronic bronchitis, sinusitis, and upper respiratory infections, including the common cold; acute and chronic gastroenteritis and colitis; acute and chronic cystitis and urethritis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; drug reactions; insect bites; burns (thermal, chemical, and electrical); and sunburn.

The present invention is also related to NO associated states. The term “NO associated state” includes states which involve or are associated with nitric oxide (NO) or inducible nitric oxide synthase (iNOS). NO associated state includes states which are characterized by aberrant amounts of NO and/or iNOS. Preferably, the NO associated state can be treated by administering tetracycline compounds of the invention (e.g., of Formula I, II-A, II-B, III, IV-A, IV-B, V, VI or Table 2). In certain embodiments, the invention includes 7-substituted, 9-substituted, 7,9-disubstituted or 10-substituted tetracyclines. The disorders, diseases and states described in U.S. Pat. Nos. 6,231,894; 6,015,804; 5,919,774; and 5,789,395 are also included as NO associated states. The entire contents of each of these patents are hereby incorporated herein by reference.

Other examples of NO associated states include, but are not limited to, malaria, senescence, diabetes, vascular stroke, neurodegenerative disorders (Alzheimer's disease & Huntington's disease), cardiac disease (re-perfusion-associated injury following infarction), juvenile diabetes, inflammatory disorders, osteoarthritis, rheumatoid arthritis, acute and chronic infections (bacterial and fungal, including diphtheria and pertussis); acute and chronic bronchitis, sinusitis, and upper respiratory infections, including the common cold; acute and chronic gastroenteritis and colitis; acute and chronic cystitis and urethritis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; drug reactions; insect bites; burns (thermal, chemical, and electrical); and sunburn.

The term “inflammatory process associated state” also includes, in one embodiment, matrix metalloproteinase associated states (MMPAS). MMPAS include states characterized by aberrant amounts of MMPs or MMP activity. These inflammatory process associated states may be treated using compounds of the invention, e.g., substituted tetracycline compounds such as those described herein (e.g., of Formula I, II-A, II-B, III, IV-A, IV-B, V, VI or Table 2).

Examples of matrix metalloproteinase associated states (“MMPAS's”) include, but are not limited to, arteriosclerosis, corneal ulceration, emphysema, osteoarthritis, multiple sclerosis (Liedtke et al., Ann. Neurol. 1998, 44:35-46; Chandler et al., J. Neuroimmunol. 1997, 72:155-71), osteosarcoma, osteomyelitis, bronchiectasis, chronic pulmonary obstructive disease, skin and eye diseases, periodontitis, osteoporosis, rheumatoid arthritis, ulcerative colitis, inflammatory disorders, tumor growth and invasion (Stetler-Stevenson et al., Annu. Rev. Cell Biol. 1993, 9:541-73; Tryggvason et al., Biochim. Biophys. Acta 1987, 907:191-217; Li et al., Mol. Carcinog. 1998, 22:84-89)), metastasis, acute lung injury, stroke, ischemia, diabetes, aortic or vascular aneurysms, skin tissue wounds, dry eye, bone and cartilage degradation (Greenwald et al., Bone 1998, 22:33-38; Ryan et al., Curr. Op. Rheumatol. 1996, 8; 238-247). Other MMPAS include those described in U.S. Pat. Nos. 5,459,135; 5,321,017; 5,308,839; 5,258,371; 4,935,412; 4,704,383, 4,666,897, and RE 34,656, incorporated herein by reference in their entirety.

The language “in combination with” another therapeutic agent or treatment includes co-administration of the tetracycline compound, (e.g., inhibitor), with the other therapeutic agent or treatment. Administration of the tetracycline compound can be provided first, followed by the other therapeutic agent or treatment. Alternatively, administration of the other therapeutic agent or treatment can be provided first, followed by the tetracycline compound. Simultaneous delivery of the tetracycline compound and the other therapeutic agent or treatment is also provided. The other therapeutic agent may be any agent which is known in the art to treat, prevent, or reduce the symptoms of an IPAS. Furthermore, the other therapeutic agent may be any agent of benefit to the patient when administered in combination with the administration of an tetracycline compound. For example, the compounds of the present invention can be administered in combination with methotrexate, dexamethasone, a steroid, or injectable biologics.

The language “effective amount” of the compound is that amount necessary or sufficient to treat or prevent an inflammatory condition, such as rheumatoid arthritis. The effective amount can vary depending on such factors as the size and weight of the subject, the type of illness, or the particular tetracycline compound. For example, the choice of the tetracycline compound can affect what constitutes an “effective amount”. One of ordinary skill in the art would be able to study the aforementioned factors and make the determination regarding the effective amount of the tetracycline compound without undue experimentation.

In the therapeutic methods of the invention, one or more tetracycline compounds of the invention may be administered alone to a subject, or more typically a compound of the invention will be administered as part of a pharmaceutical composition in mixture with conventional excipient, i.e., pharmaceutically acceptable organic or inorganic carrier substances suitable for parenteral, oral or other desired administration and which do not deleteriously react with the active compounds and are not deleterious to the recipient thereof.

### VII. Pharmaceutical Compositions

The invention also pertains to pharmaceutical compositions comprising a therapeutically effective amount of a tetracycline compound (e.g., a compound of Formula I, II-A, II-B, III, IV-A, IV-B, V, VI or Table 2) and, optionally, a pharmaceutically acceptable carrier.

The language “pharmaceutically acceptable carrier” includes substances capable of being coadministered with the tetracycline compound(s), and which allow both to perform their intended function, e.g., treat or prevent rheumatoid arthritis. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds of the invention.

The tetracycline compounds of the invention that are basic in nature are capable of forming a wide variety of salts with various inorganic and organic acids. The acids that may be used to prepare pharmaceutically acceptable acid addition salts of the tetracycline compounds of the invention that are basic in nature are those that form non-toxic acid addition salts, i.e., salts containing pharmaceutically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and palmoate [i.e., 1,1′-methylene-bis-(2-hydroxy-3-naphthoate)] salts. Although such salts must be pharmaceutically acceptable for administration to a subject, e.g., a mammal, it is often desirable in practice to initially isolate a tetracycline compound of the invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The preparation of other tetracycline compounds of the invention not specifically described in the foregoing experimental section can be accomplished using combinations of the reactions described above that will be apparent to those skilled in the art.

The preparation of other tetracycline compounds of the invention not specifically described in the foregoing experimental section can be accomplished using combinations of the reactions described above that will be apparent to those skilled in the art.

The tetracycline compounds of the invention that are acidic in nature are capable of forming a wide variety of base salts. The chemical bases that may be used as reagents to prepare pharmaceutically acceptable base salts of those tetracycline compounds of the invention that are acidic in nature are those that form non-toxic base salts with such compounds. Such non-toxic base salts include, but are not limited to those derived from such pharmaceutically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. The pharmaceutically acceptable base addition salts of tetracycline compounds of the invention that are acidic in nature may be formed with pharmaceutically acceptable cations by conventional methods. Thus, these salts may be readily prepared by treating the tetracycline compound of the invention with an aqueous solution of the desired pharmaceutically acceptable cation and evaporating the resulting solution to dryness, preferably under reduced pressure. Alternatively, a lower alkyl alcohol solution of the tetracycline compound of the invention may be mixed with an alkoxide of the desired metal and the solution subsequently evaporated to dryness.

The preparation of other tetracycline compounds of the invention not specifically described in the foregoing experimental section can be accomplished using combinations of the reactions described above that will be apparent to those skilled in the art.

The tetracycline compounds of the invention and pharmaceutically acceptable salts thereof can be administered via either the oral, parenteral or topical routes. In general, these compounds are most desirably administered in effective dosages, depending upon the weight and condition of the subject being treated and the particular route of administration chosen. Variations may occur depending upon the species of the subject being treated and its individual response to said medicament, as well as on the type of pharmaceutical formulation chosen and the time period and interval at which such administration is carried out.

The pharmaceutical compositions of the invention may be administered alone or in combination with other known compositions for treating rheumatoid arthritis in a subject, e.g., a mammal. Preferred mammals include pets (e.g., cats, dogs, ferrets, etc.), farm animals (cows, sheep, pigs, horses, goats, etc.), lab animals (rats, mice, monkeys, etc.), and primates (chimpanzees, humans, gorillas).

The tetracycline compounds of the invention may be administered alone or in combination with pharmaceutically acceptable carriers or diluents by any of the routes previously mentioned, and the administration may be carried out in single or multiple doses. For example, the novel therapeutic agents of this invention can be administered advantageously in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutically-effective compounds of this invention are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration (including intraperitoneal, subcutaneous, intravenous, intradermal or intramuscular injection), solutions of a therapeutic compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art. For parenteral application, examples of suitable preparations include solutions, preferably oily or aqueous solutions as well as suspensions, emulsions, or implants, including suppositories. Therapeutic compounds may be formulated in sterile form in multiple or single dose formats such as being dispersed in a fluid carrier such as sterile physiological saline or 5% saline dextrose solutions commonly used with injectables.

Additionally, it is also possible to administer the compounds of the present invention topically when treating inflammatory conditions of the skin. Examples of methods of topical administration include transdermal, buccal or sublingual application. For topical applications, therapeutic compounds can be suitably admixed in a pharmacologically inert topical carrier such as a gel, an ointment, a lotion or a cream. Such topical carriers include water, glycerol, alcohol, propylene glycol, fatty alcohols, triglycerides, fatty acid esters, or mineral oils. Other possible topical carriers are liquid petrolatum, isopropylpalmitate, polyethylene glycol, ethanol 95%, polyoxyethylene monolauriate 5% in water, sodium lauryl sulfate 5% in water, and the like. In addition, materials such as anti-oxidants, humectants, viscosity stabilizers and the like also may be added if desired.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

In addition to treatment of human subjects, the therapeutic methods of the invention also will have significant veterinary applications, e.g. for treatment of livestock such as cattle, sheep, goats, cows, swine and the like; poultry such as chickens, ducks, geese, turkeys and the like; horses; and pets such as dogs and cats. Also, the compounds of the invention may be used to treat non-animal subjects, such as plants.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines.

In general, compounds of the invention for treatment can be administered to a subject in dosages used in prior tetracycline therapies. See, for example, the Physicians' Desk Reference. For example, a suitable effective dose of one or more compounds of the invention will be in the range of from 0.01 to 100 milligrams per kilogram of body weight of recipient per day, preferably in the range of from 0.1 to 50 milligrams per kilogram body weight of recipient per day, more preferably in the range of 1 to 20 milligrams per kilogram body weight of recipient per day. The desired dose is suitably administered once daily, or several sub-doses, e.g. 2 to 5 sub-doses, are administered at appropriate intervals through the day, or other appropriate schedule.

It will also be understood that normal, conventionally known precautions will be taken regarding the administration of tetracyclines generally to ensure their efficacy under normal use circumstances. Especially when employed for therapeutic treatment of humans and animals in vivo, the practitioner should take all sensible precautions to avoid conventionally known contradictions and toxic effects. Thus, the conventionally recognized adverse reactions of gastrointestinal distress and inflammations, the renal toxicity, hypersensitivity reactions, changes in blood, and impairment of absorption through aluminum, calcium, and magnesium ions should be duly considered in the conventional manner.

Furthermore, the invention also pertains to the use of a tetracycline compound of Formula I, II-A, II-B, III, IV-A, IV-B, V, VI or Table 2 for the preparation of a medicament. The medicament may include a pharmaceutically acceptable carrier and the tetracycline compound in an effective amount, e.g., an effective amount to treat an inflammatory condition, such as rheumatoid arthritis.

### EXEMPLIFICATION OF THE INVENTION

Compounds of the invention may be made as described herein, with modifications to the procedures described within the skill of those of ordinary skill in the art. See e.g., Schemes 1-16 supra and characterization data in Example 4.

### Example 1

### Non-Antibacterial Derivatives of Minocycline

Minocycline derivatives (J, W, AF and AT) were tested and found to have no anti-bacterial activity compared to minocycline, and are bio-available after oral dosing in rats.

The pharmacokinetics data were acquired according to the following method:

Pre-cannulated (jugular vein and carotid artery for i.v. group and carotid artery for oral group) male CD/IGS rats (approximately 250 g) were used. Rats were fasted overnight prior to dosing with access to food restored 2 hours after dosing. Rats were administered with approximately 0.25 mL compound (1 mg/kg dose) for i.v. route (via jugular vein over 20 seconds) or 0.5 mL solution (5 mg/kg dose) orally. Blood (300 μL) was collected in tubes with EDTA anticoagulant at various time points, centrifuged and plasma collected and stored frozen at −20° C. Animals were euthanized by CO₂ following the final blood collection. Plasma was extracted (0.1% trifluoroacetic acid in 67% acetonitrile/33% water) and levels of compound quantified by HPLC/MS against a standard curve.

The results are shown in Table 3, below.

TABLE 3

| | Antibacterial Activity | | | |
|---|---|---|---|---|
| | | Protein | | |
| | | Synthesis | | |
| | E. Coli- MIC^{a} | Inhibition^{b} | PK^{c} Parameters (Rat) | |
| Compound | (μg/mL) | IC₅₀ (μM) | T_{1/2} (hr) | % F^{d} |
| Minocycline | 1 | 1.9 | 3.6 | 31 |
| W | >64 | >100 | 3.0 | 52 |
| J | >64 | >100 | 2.5 | 65 |
| AF | >64 | >100 | 3.1 | 77 |
| AT | >64 | >100 | 6.4 | 42 |

^{a}E. coli-MIC (minimal inhibitory concentration) was determined by broth micro-dilution method performed according to Clinical and Laboratory Standards Institute (CLSI) guidelines. E. coli ATCC25922 (tetracycline sensitive) was grown in cation-adjusted Mueller Hinton broth to a 0.5 McFarland standard. Turbidity was measured using a Microscan turbidity Meter.
^{b}Protein synthesis inhibition was measured using an in vitro transcription/translation assay system (E. coli S30 Extract System for Circular DNA, cat # L1020) from Promega Corporation (Madison, WI), according to the manufacturer's instructions (technical bulletin # TB092).
^{c}PK, Pharmacokinetics; All the samples were analyzed on LC-MS/MS and parameters were calculated using WinNonLin program.
^{d}% F, fraction of absorption after oral dosing of 5 mg/kg of compound.

### Example 2

### In Vivo Rheumatoid Arthritis Mouse Model

Clinical studies have demonstrated that minocycline can improve disease symptoms in rheumatoid arthritis (RA) patients. Four non-antibacterial analogues of minocycline (J, W, AF and AT) were synthesized and tested in the murine model of the disease, collagen-induced arthritis (CIA) (See supra). Male DBA/1 mice were immunized intradermally with 200 μg of bovine type II collagen and boosted with collagen three weeks later. Minocycline and four non-antibacterial minocycline derivatives were administered i.p. beginning after disease onset. Paw thickness was measured and animals were scored daily. Treatment of CIA with dexamethasone and methotrexate inhibited paw inflammation by 82% and 45% at doses of 4 mg/kg and 12 mg/kg, respectively. Minocycline inhibited the disease by 22% at 25 mg/kg/day and 45% at 50 mg/kg/day. The minocycline derivatives each inhibited CIA more potently than minocycline, ranging from 60 to 81% inhibition of paw swelling at 25 mg/kg/day. The EC₅₀ values for CIA inhibition for minocycline derivatives were lower than those of minocycline and methotrexate. Footpad tissue levels of cytokines (IL-1, IL-6, RANKL and MCP-1) and a matrix metalloproteinase (MMP-9) were decreased after therapeutic treatment of mice with dexamethasone and methotrexate, but not with minocycline. Two minocycline derivatives of the invention, however, inhibited the level of these biomarkers in the footpad tissue. These compounds may be effective for the oral treatment of RA as alternatives to commonly-used cytotoxic drugs, without the adverse effects associated with chronic administration of antibacterial drugs.

### Murine Collagen-Induced Arthritis (CIA) Model and Compound Dosing Protocol

1. Male DBA/1 mice were immunized i.d. with an emulsion of 200 μg bovine type II collagen in Complete Freund's Adjuvant.
2. On day 21, mice received i.d. boost of 100 μg collagen in Incomplete Freund's Adjuvant
3. Compounds were administered i.p. daily for 7 days starting from disease onset (day 3-4 after boost).
4. Foot swelling was measured by an engineering micrometer and disease severity was scored accordingly (1, erythema and mild swelling confined to the tarsal or ankle joint; 2, erythema and mild swelling extending from the ankle to the mid-foot; 3, erythema and moderate swelling extending from the ankle to the metatarsal joints; 4, erythema and severe swelling encompassing the ankle, foot, and digits).
5. Change (Δ) of paw thickness=sum of paw thickness from 4 paws of a mouse (experimental)−sum of baseline paw thickness from 4 paws of the same mouse.
6. % inhibition=cumulative A paw thickness (disease group−compound-treated group)/cumulative A paw thickness (disease group)

### Paw Extract Preparation and Biomarkers ELISA Assay

1. Paws were collected from mice after 5-7 days of dosing and dissected free of skin.
2. The paws were then homogenized in ice-cold PBS (2 ml/4 paws/mouse) containing 1× protease inhibitor using a Polytron homogenizer.
3. Debris and particles were removed from the homogenized samples by centrifugation.
4. Liquid layers were collected for MMP-9, IL-1, IL-6, RANKL, MCP-1, and TNFα analysis using ELISA kits from R & D System.

### Results

The minocycline derivatives J, W, AF and AT inhibited joint inflammation when administered after disease onset in a murine collagen induced arthritis (CIA) model. Effects on reduction of paw swelling and clinical score were greater when compared to either minocycline or methotrexate. Tables 4A-4G show in vivo efficacy of dexamethasone, methotrexate, minocycline, and minocycline derivatives J, W, AF and AT in reducing disease severity in the CIA model. Specifically, Table 4A shows data for dexamethasone dosed at 4 mg/kg/day i.p and the vehicle (i.p.). Table 4B shows data for methotrexate dosed at 12 mg/kg/day i.p and the vehicle (i.p.). Table 4C shows data for minocycline dosed at 25 mg/kg/day i.p and the vehicle (i.p.). Table 4D shows data for Compound W dosed at 25 mg/kg/day i.p and the vehicle (i.p.). Table 4E shows data for Compound J dosed at 25 mg/kg/day i.p and the vehicle (i.p.). Table 4F shows data for Compound AF dosed at 25 mg/kg/day i.p and the vehicle (i.p.). Table 4G shows data for Compound AT dosed at 25 mg/kg/day i.p and the vehicle (i.p.).

TABLE 4A

| | | | Days Compound Dosed | | | | | | | |
|---|---|---|---|---|---|---|---|---|---|---|
| | | | 1 | 2 | 3 | 4 | 5 | 6 | 7 | 8 |
| Change in | Vehicle | Mean | 0.89 | 1.23 | 1.68 | 1.27 | 1.43 | 1.52 | 1.66 | 1.38 |
| Paw | | SEM | 0.15 | 0.16 | 0.17 | 0.16 | 0.16 | 0.17 | 0.18 | 0.15 |
| Thickness | Dexa- | Mean | 0.92 | 0.57 | 0.47 | −0.07 | 0.03 | −0.13 | 0.04 | −0.21 |
| (mm) | methasone | SEM | 0.16 | 0.11 | 0.1 | 0.09 | 0.1 | 0.07 | 0.08 | 0.07 |

TABLE 4B

| | | | Days Compound Dosed | | | | | | | |
|---|---|---|---|---|---|---|---|---|---|---|
| | | | 1 | 2 | 3 | 4 | 5 | 6 | 7 | 8 |
| Change in | Vehicle | Mean | 0.71 | 0.78 | 0.93 | 1.03 | 1.29 | 1.3 | 1.26 | 1.46 |
| Paw | | SEM | 0.12 | 0.13 | 0.16 | 0.14 | 0.14 | 0.14 | 0.13 | 0.17 |
| Thickness | Metho- | Mean | 0.77 | 0.68 | 0.71 | 0.68 | 0.67 | 0.56 | 0.45 | 0.28 |
| (mm) | trexate | SEM | 0.13 | 0.1 | 0.1 | 0.1 | 0.1 | 0.1 | 0.08 | 0.08 |

TABLE 4C

| | | | Days Compound Dosed | | | | | | | |
|---|---|---|---|---|---|---|---|---|---|---|
| | | | 1 | 2 | 3 | 4 | 5 | 6 | 7 | 8 |
| Change in | Vehicle | Mean | 0.69 | 0.69 | 0.93 | 1.07 | 1.28 | 1.46 | 1.47 | 1.73 |
| Paw | | SEM | 0.08 | 0.09 | 0.1 | 0.1 | 0.11 | 0.11 | 0.11 | 0.13 |
| Thickness | Mino- | Mean | 0.74 | 0.72 | 0.78 | 0.86 | 0.93 | 1.07 | 0.92 | 1.18 |
| (mm) | cycline | SEM | 0.11 | 0.12 | 0.11 | 0.11 | 0.11 | 0.11 | 0.11 | 0.13 |

TABLE 4D

| | | | Days Compound Dosed | | | | | | | |
|---|---|---|---|---|---|---|---|---|---|---|
| | | | 1 | 2 | 3 | 4 | 5 | 6 | 7 | 8 |
| Change in | Vehicle | Mean | 0.68 | 0.74 | 1.04 | 1.13 | 1.27 | 1.4 | 1.49 | 1.55 |
| Paw | | SEM | 0.05 | 0.06 | 0.07 | 0.06 | 0.07 | 0.07 | 0.07 | 0.08 |
| Thickness | Compound W | Mean | 0.71 | 0.41 | 0.46 | 0.56 | 0.51 | 0.45 | 0.35 | 0.60 |
| (mm) | | SEM | 0.09 | 0.07 | 0.1 | 0.08 | 0.07 | 0.08 | 0.09 | 0.11 |

TABLE 4E

| | | | Days Compound Dosed | | | | | | | |
|---|---|---|---|---|---|---|---|---|---|---|
| | | | 1 | 2 | 3 | 4 | 5 | 6 | 7 | 8 |
| Change in | Vehicle | Mean | 0.68 | 0.74 | 1.04 | 1.13 | 1.27 | 1.4 | 1.49 | 1.55 |
| Paw | | SEM | 0.05 | 0.06 | 0.07 | 0.06 | 0.07 | 0.07 | 0.07 | 0.08 |
| Thickness | Compound J | Mean | 0.47 | 0.39 | 0.33 | 0.48 | 0.36 | 0.37 | 0.50 | 0.65 |
| (mm) | | SEM | 0.07 | 0.07 | 0.1 | 0.08 | 0.08 | 0.08 | 0.11 | 0.12 |

TABLE 4F

| | | | Days Compound Dosed | | | | | | | |
|---|---|---|---|---|---|---|---|---|---|---|
| | | | 1 | 2 | 3 | 4 | 5 | 6 | 7 | 8 |
| Change in | Vehicle | Mean | 0.68 | 0.74 | 1.04 | 1.13 | 1.27 | 1.4 | 1.49 | 1.55 |
| Paw | | SEM | 0.05 | 0.06 | 0.07 | 0.06 | 0.07 | 0.07 | 0.07 | 0.08 |
| Thickness | Compound | Mean | 0.55 | 0.39 | 0.61 | 0.49 | 0.46 | 0.38 | 0.42 | 0.54 |
| (mm) | AF | SEM | 0.07 | 0.06 | 0.09 | 0.07 | 0.08 | 0.06 | 0.07 | 0.14 |

TABLE 4G

| | | | Days Compound Dosed | | | | | | | |
|---|---|---|---|---|---|---|---|---|---|---|
| | | | 1 | 2 | 3 | 4 | 5 | 6 | 7 | 8 |
| Change in | Vehicle | Mean | 0.68 | 0.74 | 1.04 | 1.13 | 1.27 | 1.4 | 1.49 | 1.55 |
| Paw | | SEM | 0.05 | 0.06 | 0.07 | 0.06 | 0.07 | 0.07 | 0.07 | 0.08 |
| Thickness | Compound | Mean | 0.94 | 0.57 | 0.55 | 0.11 | 0.05 | 0.27 | −0.01 | −0.08 |
| (mm) | AT | SEM | 0.12 | 0.09 | 0.08 | 0.06 | 0.1 | 0.06 | 0.08 | 0.07 |

In Tables 4A-4G, SEM = standard error of the mean

Table 5 shows a comparison of disease severity (paw swelling and clinical score) in CIA mice that were treated with minocycline vs. minocycline derivatives. Table 6, below, shows a comparison of EC₅₀ values of minocycline and several minocycline derivatives in inflammation suppression in CIA mice.

TABLE 5

| | | |
|---|---|---|
| | % Inhibition | |
| | (Change in Paw | % Inhibition |
| Compound | Thickness) | (Clinical Score) |

| (Dose) | AVG | SD | AVG | SD |
|---|---|---|---|---|
| Minocycline | 22 | 12 | 18 | 7 |
| (25 mg/kg/day | | | | |
| i.p.) | | | | |
| Compound W | 60 | 12 | 40 | 5 |
| (25 mg/kg/day | | | | |
| i.p.) | | | | |
| Compound J | 71 | 16 | 45 | 2 |
| (25 mg/kg/day | | | | |
| i.p.) | | | | |
| Compound AF | 62 | 16 | 42 | 14 |
| (25 mg/kg/day | | | | |
| i.p.) | | | | |
| Compound AT | 81 | 13 | 70 | 10 |
| (25 mg/kg/day | | | | |
| i.p.) | | | | |
| Dexamethasone | 82 | 21 | 48 | 18 |
| (4 mg/kg) | | | | |
| Methotrexate | 45 | 16 | 25 | 13 |
| (12 mg/kg) | | | | |

In Table 5, AVG = average and SD = standard deviation

TABLE 6

| Compounds | EC₅₀ (mg/kg/day i.p.) |
|---|---|
| Minocycline | >50 |
| W | 20 |
| J | 14 |
| AF | 12 |
| AT | 12 |

The minocycline derivatives J, W, AF and AT inhibited inflammatory/osteoclastic cytokines (MMP-9, IL-1, IL-6, MCP-1, RANKL) better than minocycline in vivo. Table 7 shows enzyme-linked immunosorbent assay (ELISA) analysis of inflammatory biomarkers using paw extracts from CIA mice. Table 8 shows a comparison of inflammatory biomarker expression in paws of CIA mice treated with several compounds.

TABLE 7

| | |
|---|---|
| Naïve | |
| (Tissue [c] pg/mL) | CIA |

| Biomarker | AVG | SD | AVG | SD |
|---|---|---|---|---|
| MMP-9 | 8 | 4 | 60 | 17 |
| IL-1 | 14 | 3 | 306 | 69 |
| IL-6 | 28 | 5 | 174 | 32 |
| RANKL | 13 | 4 | 264 | 41 |
| MCP-1 | 104 | 18 | 687 | 173 |
| TNFα | 4 | 1 | 5 | 2 |

TABLE 8

| | Percent level of Biomarker Tissue [c] Compared to Untreated Control | | | | | | | | | |
|---|---|---|---|---|---|---|---|---|---|---|
| | MMP-9 | | IL-1 | | IL-6 | | MCP-1 | | RANKL | |
| Compound | AVG | SD | AVG | SD | AVG | SD | AVG | SD | AVG | SD |
| Dexamethasone | 35 | 8 | 16 | 7 | 4 | 2 | 5 | 3 | 10 | 5 |
| Methotrexate | 52 | 26 | 16 | 10 | 20 | 11 | 37 | 23 | — | — |
| Minocycline | 104 | 8 | 119 | 16 | 114 | 38 | 102 | 24 | 99 | 15 |
| Compound W | 72 | 14 | 32 | 10 | 28 | 12 | 28 | 12 | 27 | 11 |
| Compound J | 68 | 15 | 52 | 14 | 68 | 24 | 60 | 20 | 32 | 22 |
| Compound AF | 113 | 15 | 55 | 15 | 35 | 15 | 43 | 11 | 38 | 15 |

In Tables 7-8, AVG = average and SD = standard deviation

### Example 3

### A Study of the Inhibition of Collagen-Induced Arthritis and Antibacterial Activity of Various Tetracycline Compounds

Several substituted tetracycline compounds were tested for antibacterial activity and inhibition of the CIA model. The results are shown in Table 9.

The antibacterial activity were acquired according to the following method:

2 mg of each compound is dissolved in 100 μl of DMSO. The solution is then added to cation-adjusted Mueller Hinton broth (CAMHB), which results in a final compound concentration of 200 μg per ml. The tetracycline compound solutions are diluted to 50 μL volumes, with a test compound concentration of 0.098 μg/ml. Optical density (OD) determinations are made from fresh log-phase broth cultures of the test strains. Dilutions are made to achieve a final cell density of 1×10⁶ CFU/ml. At OD=1, cell densities for different genera should be approximately:

| | |
|---|---|
| E. coli | 1 × 10⁹ CFU/ml |
| S. aureus | 5 × 10⁸ CFU/ml |
| Enterococcus sp. | 2.5 × 10⁹ CFU/ml |

50 μl of the cell suspensions are added to each well of microtiter plates. The final cell density should be approximately 5×10⁵ CFU/ml. These plates are incubated at 35° C. in an ambient air incubator for approximately 18 hr. The plates are read with a microplate reader and are visually inspected when necessary. The MIC is defined as the lowest concentration of the tetracycline compound that inhibits growth.

The CIA model data were acquired according to the following protocol:
1. Male DBA/1 mice were anaesthetized by i.p. injection of ketamine/xylazine (1.25 mg/ml:0.25 mg/ml, 100 μl/mouse).
2. Anaesthetized mice were immunized intradermally at the base of the tail with 0.1 or 0.2 ml of an emulsion composed of 100 or 200 μg bovine type II collagen in Complete Freund's Adjuvant (50 μl/spot x 2 or 4 spots).
3. At day 21 after the immunization, mice received an intradermal boost of 100 μl of an emulsion composed of 100 μg bovine type II collagen in Incomplete Freund's Adjuvant (50 μl/spot x 2 spots)
4. After onset of disease symptoms (usually 3-4 days after boost), tetracycline derivatives were administered daily into mice via intraperitoneal or oral routes for 8 to 15 days. Methotrexate (12 mg/kg/day) or dexmethasone (4 mg/kg/day) was used as a control for inflammation suppression.
5. The disease severity was scored daily for 8-15 days after the appearance of the symptoms.
   Disease severity was graded as following:
a. Visual clinical score for the presence of inflammation in the fingers/toes of the forepaws and hindpaws:
   1. Erythema and mild swelling confined to the mid-foot (tarsals) or ankle joint
   2. Erythema and mild swelling extending from the ankle to the mid-foot
   3. Erythema and moderate swelling extending from the ankle to the metatarsal joints
   4. Erythema and severe swelling encompassing the ankle, foot, and digits.
b. Clinical swelling score for the presence of paw edema in the forepaws and hindpaws:
   Paw thickness was measured daily by an engineering micrometer after isofluorane inhalation or ketamine/xylazine (1.25 mg/ml:0.25 mg/ml, 100 μl/mouse) i.p. injection.

Daily scores and paw swelling measurements for each treatment group of mice were added over the total observation period to obtain a cumulative score. Cumulative scores were compared between untreated controls and treated groups to determine tetracycline-induced inhibition.

TABLE 9

| | Antibacterial Activity | | |
|---|---|---|---|
| | MIC (μg/mL) | | CIA Model |
| | Gram+ | Gram− | % Inhibition |
| Compound | (S. aureus RN450) | (E. coli 25922) | (Dose mg/kg) |
| Minocycline | 0.06 | 1 | 22 (25) |
| A | 1 | >64 | 42 (25) |
| B | 0.13 | 64 | 23 (25) |
| C | 0.06 | 0.13 | 62 (25) |
| | | | 62 (12) |
| D | — | — | 27 (25) |
| E | 2 | >64 | 44 (25) |
| F | — | — | 55 (25) |
| G | 0.5 | 16 | 55 (25) |
| H | 0.5 | >64 | 41 (25) |
| I | — | — | 29 (25) |
| J | 2 | >64 | 71 (25) |
| | | | 34 (12) |
| K | 4 | >64 | 30 (25) |
| L | 2 | 64 | 48 (25) |
| M | 8 | >64 | 25 (25) |
| N | 0.06 | 0.5 | 21 (25) |
| O | 32 | >64 | 26 (25) |
| P | 0.5 | >64 | 34 (25) |
| Q | 0.5 | 32 | 28 (25) |
| R | 2 | >64 | 32 (25) |
| S | >64 | >64 | 46 (25) |
| T | 4 | >64 | 44 (25) |
| | | | 10 (12) |
| U | >64 | >64 | 50 (25) |
| V | 2 | >64 | 43 (25) |
| W | 4 | >64 | 60 (25) |
| | | | 32 (12) |
| | | | 25 (6) |
| X | 2 | >64 | 40 (25) |
| Y | >64 | >64 | 22 (25) |
| Z | 4 | >64 | 32 (25) |
| AA | 2 | >64 | 41 (25) |
| AB | 0.5 | >64 | 38 (25) |
| AC | 2 | >64 | 33 (25) |
| AD | 2 | >64 | 30 (25) |
| AE | 1 | >64 | 80 (25) |
| | | | 65 (12) |
| AF | 1 | >64 | 62 (35) |
| | | | 49 (12) |
| | | | 28 (6) |
| AG | 0.25 | >64 | 39 (25) |
| AH | 1 | >64 | 59 (25) |
| AI | 0.12 | >64 | 62 (25) |
| AJ | 0.25 | >64 | 39 (25) |
| AK | >64 | >64 | 24 (25) |
| AL | 1 | >64 | 62 (25) |
| | | | 31 (12) |
| AM | 2 | >64 | 73 (25) |
| | | | 66 (12) |
| | | | 36 (6) |
| AN | 1 | >64 | 65 (25) |
| | | | 49 (12) |
| AO | 16 | >64 | 52 (25) |
| AP | 4 | >64 | 63 (25) |
| AQ | 1 | >64 | 76 (25) |
| | | | 62 (12) |
| AR | 0.5 | >64 | 64 (25) |
| AS | 1 | >64 | 56 (25) |
| | | | 30 (12) |
| AT | 1 | >64 | 81 (25) |
| | | | 49 (12) |
| | | | 45 (6) |
| AU | 1 | >64 | 51 (25) |
| AV | 1 | >64 | 48 (25) |
| AW | 0.5 | >64 | 39 (25) |
| AX | 0.5 | >64 | 51 (25) |
| AY | 8 | >64 | 38 (25) |
| AZ | 1 | >64 | 36 (25) |
| BA | 0.25 | 64 | 34 (25) |
| BB | 8 | >64 | 47 (25) |
| BC | 4 | >64 | 72 (25) |
| BD | 0.5 | >64 | 44 (25) |

### Example 4

### Physicochemical Data for Several Substituted Tetracycline Compounds

Table 10, below, shows LCMS and ¹H NMR data for several compound of the present invention.

TABLE 10

| | LCMS | |
|---|---|---|
| Com- | (Obs. | |
| pound | m/z of | ¹H-NMR (300 MHz, CD₃OD, ppm rel. to CH₃OH = |
| ID | MH+) | 3.34 ppm) |
| A | 534 | δ 7.85 (s, 1H), 7.58 (m, 2H), 7.40 (m, 2H), 4.15 (s, |
| | | 1H), 3.42 (m, 1H), 2.98 (dd, 6H), 2.57 (dd, 1H), 2.30 |
| | | (dm, 1H), 1.62 (m, 1H) |
| B | 535 | δ 1.45-1.65 (m, 1H), 2.0-2.15 (m, 1H), 2.35-2.55 (m, |
| | | 1H), 2.8-2.95 (m, 9H), 4.05 (s, 1H), 6.0 (s, 2H), 6.5- |
| | | 6.7 (m, 2H) 6.9-7.0 (m, 2H), 7.35-7.45 (m, 1H) |
| C | 457 | δ 1.4-1.65 (m, 1H), 1.9-2.2 (m, 1H) 2.3-2.6 (m, 4H), |
| | | 2.7-3.05 (m, 7H), 3.05-3.2 (m, 1H), 3.4-3.6 (m, 1H), |
| | | 4.05 (s, 1H), 6.8-6.95 (m, 1H), 7.85-8.0 (m, 1H) |
| D | 581 | δ 1.4-1.6 (m, 1H), 1.8-1.9 (m, 0.5H), 2.0-2.1 (m, |
| | | 0.5H), 2.4-2.6 (m, 1H), 2.7-3.2 (m, 9H), 3.7-3.8 (m, |
| | | 9H), 4.0 (s, 0.5H), 6.5-6.53 (m, 2H), 6.8-6.9 (m, 1H) |
| | | 7.4-7.5 (m, 1H) |
| E | 500 | δ 0.95-1.05 (m, 2H), 1.55-1.8 (m, 2H), 2.05-2.3 (m, |
| | | 1H) 2.35-2.55 (m, 1H), 2.6-2.8 (m, 2H), 2.9-3.15 (m, |
| | | 16H), 3.15-3.5 (m, 1H), 4.1-4.15 (m, 0.5H), 7.7 (s, |
| | | 1H) |
| G | 473 | δ 7.38 (d, 1H), 6.85 (d, 1H), 4.41 (s, 1H), 3.55 (m, |
| | | 1H), 2.95 (s, 6H), 2.70 (m, 5H), 1.55 (d, 3H), 1.20 (t, |
| | | 3H) |
| H | 486 | δ 7.69 (s, 1H), 4.05 (s, 1H), 2.90 (m, 7H), 2.61 (q, |
| | | 2H), 2.40 (m, 1H), 2.21 (dm, 1H), 1.55 (m, 1H), 1.14 |
| | | (t, 3H) |
| I | 498 | δ 1.5-1.8 (m, 1H), 2.1-2.4 (m, 1H) 2.4-2.6 (m, 1H), |
| | | 2.9-3.15 (m, 6H), 3.2-3.4 (m, 7H), 3.4-3.6 (m, 2H), |
| | | 4.16 (s, 1H), 5.0-5.2 (m, 2H), 5.9-6.2 (m, 1H), 7.6 (s, |
| | | 1H) |
| J | 500 | δ 7.80 (s, 1H), 4.20 (s, 1H), 3.45 (m, 7H), 3.25 (m, |
| | | 3H), 3.05 (m, 8H), 2.53 (m, 1H), 2.36 (dm, 1H), 1.32 |
| | | (m, 6H) |
| K | 529 | δ 7.34 (s, 1H), 3.93 (s, 3H), 3.68 (t, 1H), 3.38 (m, |
| | | 1H), 2.90 (m, 2H), 2.72 (s, 6H), 2.59 (s, 6H), 2.20 |
| | | (m, 4H), 2.20 (m, 1H), 1.65 (m, 1H) |
| L | 496 | δ 1.4-1.7 (m, 1H), 2.05-2.2 (m, 1H) 2.2-2.45 (m, 2H), |
| | | 2.5-2.65 (m, 1H), 2.8-3.0 (m, 8H), 3.3-3.45 (m, 2H), |
| | | 3.7-3.8 (m, 2H), 4.01 (s, 1H) 5.5-5.7 (m, 1H), 6.7-6.8 |
| | | (m, 1H), 7.15-7.3 (m, 1H) |
| M | 514 | δ 8.23 (s, 1H), 4.12 (s, 1H), 3.38 (m, 1H), 3.25 (m, |
| | | 6H), 3.10 (m, 4H), 3.00 (m, 6H), 2.51 (m, 1H), 2.23 |
| | | (dm, 1H), 1.62 (m, 1H), 1.11 (t, 3H) |
| N | 507 | δ 1.49-1.8 (m, 1H), 2.0-2.25 (m, 1H), 2.35-2.7 (m, |
| | | 1H), 2.8-3.25 (m, 9H), 4.1 (s, 1H), 3.38 (s, 1H), 6.57- |
| | | 6.80 (m, 3H) 7.0-7.2 (m, 2H), 7.3-7.41 (m, 1H) |
| O | 525 | δ 8.59 (s, 1H), 8.36 (s, 1H), 7.74 (s, 1H), 4.19 (s, |
| | | 1H), 3.50 (m, 1H), 3.38 (m, 7H), 3.22 (m, 1H), 3.05 |
| | | (m, 7H), 2.65 (m, 1H), 2.36 (dm, 1H), 1.70 (m, 1H) |
| P | 540 | δ 1.55-1.8 (m, 1H), 2.1-2.35 (m, 1H) 2.4-2.55 (m, |
| | | 1H), 2.9-3.15 (m, 7H), 3.15-3.5 (m, 9H), 4.13 (s, |
| | | 0.5H), 4.85-4.9 (m, 0.5H), 7.45-7.55 (m, 1H), 7.6-7.7 |
| | | (m, 1H) 7.9-8.0 (m, 1H), 8.05-8.1 (m, 1H) |
| Q | 399 | δ 7.95 (d, 1H), 7.50 (m, 1H), 7.38 (m, 1H), 7.28 (d, |
| | | 1H), 4.05 (s, 1H), 3.05 (m, 8H), 2.82 (m, 1H), 2.60 |
| | | (m, 1H), 2.15 (m, 1H), 1.65 (m, 1H) |
| R | 577 | δ 7.98 (s, 1H), 7.85 (m, 2H), 7.70 (m, 2H), 4.15 (s, |
| | | 1H), 3.40 (m, 1H), 3.25 (m, 6H), 3.10 (m, 1H), 3.00 |
| | | (dm, 6H), 2.60 (m, 1H), 2.31 (dm, 1H), 1.65 (m, 1H) |
| S | 577 | δ 7.91 (m, 3H), 7.69 (m, 2H), 4.12 (s, 1H), 3.36 (m, |
| | | 1H), 3.25 (m, 6H), 3.15 (m, 1H), 2.96 (dm, 6H), 2.51 |
| | | (m, 1H), 2.28 (dm, 1H), 1.61 (m, 1H) |
| T | 457 | δ 8.30 (s, 1H), 3.25 (m, 1H), 3.00 (m, 1H), 2.70 (s, |
| | | 3H), 2.50 (m, 3H), 2.13 (dm, 1H), 1.67 (m, 1H) |
| U | 459 | δ 8.35 (s, 1H), 3.38 (s, 1H), 3.25 (m, 6H), 3.21 (m, |
| | | 1H), 2.95 (m, 1H), 2.49 (m, 3H), 2.15 (dm, 1H), 1.65 |
| | | (m, 1H) |
| V | 500 | δ 8.45 (s, 1H), 3.42 (t, 2H), 3.15 (dd, 1H), 3.00 (m, |
| | | 1H), 2.51 (m, 3H), 2.15 (dm, 1H), 1.66 (m, 1H), 1.00 |
| | | (t, 3H) |
| W | 473 | δ 8.30 (s, 1H), 3.95 (s, 3H), 3.36 (s, 1H), 3.24 (m, |
| | | 8H), 2.98 (m, 1H), 2.50 (m, 3H), 2.15 (dm, 1H), 1.66 |
| | | (m, 1H) |
| X | 482 | δ 8.50 (s, 1H), 8.40 (s, 1H), 7.80 (s, 1H), 3.40 (m, |
| | | 7H), 3.25 (m, 1H), 3.05 (m, 1H), 2.55 (m, 3H), 2.17 |
| | | (dm, 1H), 1.70 (m, 1H) |
| Y | 526 | δ 1.55-1.8 (m, 7H), 2.1-2.2 (m, 1H) 2.3-2.7 (m, 3H), |
| | | 2.95-3.1 (m, 1H), 3.15-3.25 (m, 2H), 3.25-3.4 (m, |
| | | 7H), 3.6-3.85 (m, 2H), 7.95 (s, 1H) |
| Z | 543 | δ 7.34 (s, 1H), 4.18 (q, 2H), 3.68 (t, 1H), 3.36 (m, |
| | | 1H), 2.90 (m, 2H), 2.87 (s, 6H), 2.59 (s, 6H), 2.20 (s, |
| | | 3H), 2.19 (m, 2H), 2.61 (q, 1H), 1.30 (t, 3H) |
| AA | 487 | δ 8.30 (s, 1H), 4.41 (q, 2H), 3.37 (s, 1H), 3.29 (m, |
| | | 6H), 3.23 (m, 2H), 2.98 (m, 1H), 2.50 (m, 3H), 2.15 |
| | | (dm, 1H), 1.68 (m, 1H), 1.42 (t, 3H) |
| AB | 482 | δ 9.35 (s, 1H), 9.20 (s, 1H), 8.31 (s, 1H), 3.38 (m, |
| | | 6H), 3.21 (m, 2H), 3.02 (m, 1H), 2.51 (m, 3H), 2.18 |
| | | (dm, 1H), 1.70 (m, 1H) |
| AC | 481 | δ 1.6-1.8 (m, 1H), 2.1-2.3 (m, 1H) 2.35-2.7 (m, 3H), |
| | | 2.95-3.15 (m, 1H), 3.2-3.3 (m, 1H), 3.37-3.5 (m, 7H), |
| | | 7.3-7.4 (m, 1H), 8.15-8.25 (m, 1H), 8.51 (s, 1H) |
| AD | 486 | δ 7.34 (s, 1H), 3.93 (s, 3H), 3.38 (dd, 1H), 3.26 (m, |
| | | 1H), 2.75 (m, 1H), 2.58 (s, 6H), 2.47 (m, 2H), 2.19 |
| | | (s, 3H), 2.06 (m, 2H), 1.60 (q, 1H) |
| AE | 492 | δ 9.32 (s, 1H), 9.05 (m, 1H), 8.92 (m, 1H), 8.31 (s, |
| | | 1H), 8.25 (m, 1H), 3.38 (m, 8H), 3.15 (m, 1H), 2.56 |
| | | (m, 3H), 2.20 (dm, 1H), 1.61 (m, 1H) |
| AF | 443 | δ 7.78 (s, 1H), 3.18 (m, 4H), 2.95 (m, 1H), 2.73 (q, |
| | | 2H), 2.43 (m, 3H), 2.11 (dm, 1H), 1.64 (m, 1H), 1.24 |
| | | (t, 3H) |
| AG | 481 | δ 8.29 (s, 1H), 7.65 (t, 1H), 7.20 (d, 1H), 6.61 (m, |
| | | 1H), 3.24 (d, 1H), 3.14 (dd, 1H), 2.98 (m, 1H), 2.49 |
| | | (m, 3H), 2.13(dm, 1H), 1.65 (m, 1H) |
| AH | 514 | δ 1.4 (s, 9H), 1.6-1.8 (m, 1H), 2.1-2.25 (m, 1H), 2.35- |
| | | 2.7 (m, 3H), 2.9-3.1 (m, 1H), 3.15-3.3 (m, 1H), 3.38 |
| | | (s, 1H), 8.45 (s, 1H) |
| AI | 534 | δ 8.47 (s, 1H), 7.71 (m, 2H), 7.38 (m, 2H), 7.18 (m, |
| | | 1H), 3.38 (s, 1H), 3.26 (m, 6H), 3.21 (m, 1H), 3.00 |
| | | (m, 1H), 2.49 (m, 3H), 2.12 (dm, 1H), 1.66 (m, 1H) |
| AJ | 498 | δ 9.05 (s, 1H), 8.15 (s, 1H), 7.98 (s, 1H), 3.40 (m, |
| | | 6H), 3.35 (s, 1H), 3.29 (m, 1H), 3.05 (m, 1H), 2.51 |
| | | (m, 3H), 2.20 (dm, 1H), 1.70 (m, 1H) |
| AK | 503 | δ 8.46 (s, 1H), 4.43 (m, 2H), 3.91 (m, 2H), 3.36 (m, |
| | | 6H), 3.22 (m, 2H), 3.01 (m, 1H), 2.52 (m, 3H), 2.15 |
| | | (dm, 1H), 1.68 (m, 1H) |
| AL | 471 | δ 8.25 (s, 1H), 3.13 (m, 3H), 2.98 (m, 1H), 2.50 (m, |
| | | 3H), 2.12(dm, 1H), 1.68 (m, 1H), 1.17 (t, 3H) |
| AM | 429 | δ 7.80 (s, 1H), 3.25 (m, 6H), 3.12 (m, 1H), 2.96 (m, |
| | | 1H), 2.50 (m, 3H), 2.30 (s, 3H), 2.13 (dm, 1H), 1.69 |
| | | (m, 1H) |
| AN | 491 | δ 7.91 (s, 1H), 7.61 (m, 2H), 7.42 (m, 3H), 3.19 (m, |
| | | 2H), 2.98 (m, 1H), 2.45 (m, 3H), 2.14 (dm, 1H), 1.65 |
| | | (m, 1H) |
| AO | 517 | δ 1.55-1.8 (m, 1H), 2.1-2.2 (m, 1H) 2.3-2.6 (m, 3H), |
| | | 2.85-3.0 (m, 1H), 3.0-3.2 (m, 6H), 3.2-3.4 (m, 2H), |
| | | 3.45 (s, 3H), 3.65-3.8 (m, 2M), 4.4-4.45 (m, 2H), |
| | | 8.21 (s, 1H) |
| AP | 497 | δ 7.56 (s, 1H), 5.70 (t, 1H), 4.12 (m, 2H), 3.01 (m, |
| | | 1H), 2.72 (m, 1H), 2.56 (s, 6H), 2.36 (m, 2H), 1.99- |
| | | 2.3 (m, 5H), 1.94 (m, 2H), 1.5-1.85 (m, 2H) |
| AQ | 414 | δ 7.42 (d, 1H), 6.77 (d, 1H), 2.79 (m, 1H), 2.29 (m, |
| | | 3H), 2.02 (m, 1H), 1.58 (m, 1H), 1.16 (dd, 6H) |
| AR | 525 | δ 1.47 (d, J = 7.5 Hz, 6H), 1.55-1.75 (m, 1H), 2.0-2.2 |
| | | (m, 1H), 2.15-2.6 (m, 3H), 2.6-2.9 (m, 7H), 3.05-3.19 |
| | | (m, 1H), 3.20-3.45 (m, 3H), 8.00 (s, 1H) |
| AS | 495 | δ 1.6-1.8 (m, 1H), 2.1-2.25 (m, 1H), 2.35-2.65 (m, |
| | | 3H), 2.8-3.2 (m, 6H), 3.2-3.3 (m, 1H), 3.79 (s, 3H), |
| | | 6.4 (s, 1H), 7.55 (s, 1H), 7.75 (brs, 1H) |
| AT | 494 | δ 1.6-1.8 (m, 1H), 2.05-2.2 (m, 1H) 2.33-2.65 (m, |
| | | 3H), 2.9-3.1 (m, 1H), 3.1-3.29 (m, 8H), 3.51 (m, 3H), |
| | | 6.01-6.2 (m, 2H), 6.72-6.85 (m, 1H), 7.75 (s, 1H) |
| AU | 457 | δ 7.73 (1H), 3.11 (m, 1H), 2.94 (m, 1H), 2.43 (m, |
| | | 3H), 2.11 (dm, 1H), 1.63 (m, 1H), 1.26 (m, 6H) |
| AV | 455 | δ 0.70-0.90)m, 1H), 0.91-1.15 (m, 1H), 1.58-1.80 |
| | | (m, 1H), 2.00-2.40 (m, 2H), 2.40-2.65 (m, 3H), 2.80- |
| | | 3.10 (s, 1H), 3.10-3.40 (brm, 8H), 7.45 (s, 1H) |
| AW | 485 | δ 7.69 (s, 1H), 3.11 (dd, 1H), 2.96 (m, 1H), 2.66 (m, |
| | | 2H), 2.45 (m, 3H), 2.12 (dm, 1H), 1.65 (m, 1H), 0.96 |
| | | (s, 9H) |
| AX | 513 | δ 8.16 (s, 1H), 3.18 (m, 1H), 3.07 (s, 2H), 2.97 (m, |
| | | 1H), 2.50 (m, 3H), 2.12 (dm, 1H), 1.66 (m, 1H), 1.06 |
| | | (s, 9H) |
| AY | 413 | δ 7.85 (d, 1H), 7.45 (d, 1H), 3.20 (m, 2H), 2.89 (m, |
| | | 1H), 2.73 (s, 3H), 2.48 (m, 3H), 2.18 (dm, 1H), 1.66 |
| | | (m, 1H) |
| AZ | 519 | δ 8.02 (s, 1H), 7.83 (m, 2H), 7.65 (m, 1H), 7.50 (m, |
| | | 2H), 3.18 (dd, 1H), 3.04 (m, 1H), 2.51 (m, 3H), 2.15 |
| | | (dm, 1H), 1.70 (m, 1H) |
| BA | 386 | δ 7.26 (d, 1H), 6.67 (d, 1H), 2.98 (dd, 1H), 2.79 (m, |
| | | 1H), 2.44 (m, 2H), 2.20 (m, 4H), 2.01 (m, 1H), 1.56 |
| | | (m, 1H) |
| BB | 439 | δ 8.03 (s, 1H), 7.96 (d, 1H), 7.36 (s, 1H), 6.96 (d, |
| | | 1H), 3.64 (m, 1H), 2.84 (m, 1H), 2.48 (m, 3H), 2.02 |
| | | (m, 1H), 1.59 (m, 1H) |
| BC | 414 | δ 7.96 (d, 1H), 6.87 (d, 1H), 3.45 (dd, 1H), 2.78 (m, |
| | | 1H), 2.52 (s, 3H), 2.41 (m, 3H), 1.99 (dm, 1H), 1.55 |
| | | (m, 1H) |

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

What is claimed is:

1. A method for treating rheumatoid arthritis in a subject, comprising administering to the subject a tetracycline compound of Formula I: 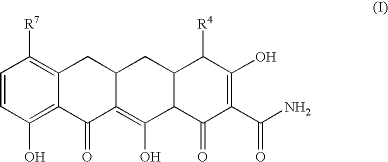
wherein:
R⁴ is amino or hydrogen; and
R⁷ is substituted or unsubstituted alkyl, substituted or unsubstituted heterocyclyl, substituted or unsubstituted phenyl, substituted or unsubstituted heteroaryl, or substituted or unsubstituted acyl;
or a pharmaceutically acceptable salt, ester or prodrug thereof;
such that the rheumatoid arthritis is treated in the subject.

2. The method of claim 1, wherein R⁴ is dimethylamino.

3. The method of claim 1, wherein R⁴ is hydrogen.

4. The method of claim 1, wherein R⁷ is substituted or unsubstituted acyl.

5. The method of claim 1, wherein R⁷ is substituted or unsubstituted phenyl.

6. A method for treating rheumatoid arthritis in a subject, comprising administering to the subject a tetracycline compound of Formula III: 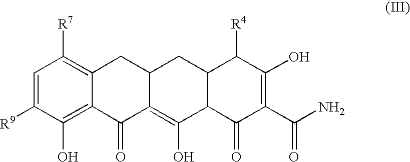
wherein:
R⁴ is amino or hydrogen;
R⁷ is amino or hydrogen; and
R⁹ is substituted or unsubstituted alkyl, substituted or unsubstituted heterocyclyl, substituted or unsubstituted phenyl, substituted or unsubstituted heteroaryl, substituted or unsubstituted acyl, or substituted or unsubstituted imine;
or a pharmaceutically acceptable salt, ester or prodrug thereof;
such that the rheumatoid arthritis is treated in the subject.

7. The method of claim 6, wherein R⁴ is dimethylamino and R⁷ is dimethylamino.

8. The method of claim 6, wherein R⁴ is hydrogen and R⁷ is dimethylamino.

9. The method of claim 6, wherein R⁹ is substituted or unsubstituted C₁-C₅ alkyl.

10. The method of claim 9, wherein R⁹ is unsubstituted C₂-C₄ alkyl.

11. The method of claim 6, wherein R⁹ is substituted or unsubstituted heteroaryl.

12. The method of claim 11, wherein R⁹ is substituted pyrrolyl.

13. The method of claim 6, wherein R⁹ is substituted or unsubstituted acyl.

14. The method of claim 13, wherein R⁹ is alkoxy substituted acyl.

15. A method for treating rheumatoid arthritis in a subject, comprising administering to the subject a tetracycline compound of Formula V: 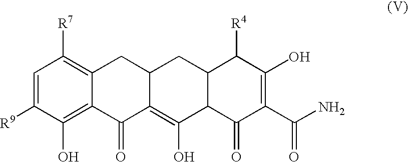
wherein:
R⁴ is amino or hydrogen;
R⁷ is substituted or unsubstituted C₁-C₅ alkyl, substituted or unsubstituted heterocyclyl, substituted or unsubstituted phenyl, substituted or unsubstituted heteroaryl, substituted or unsubstituted acyl; and
R⁹ is substituted or unsubstituted C₁-C₅ alkyl, substituted or unsubstituted heterocyclyl, substituted or unsubstituted phenyl, substituted or unsubstituted heteroaryl, substituted or unsubstituted acyl, or substituted or unsubstituted imine;
or a pharmaceutically acceptable salt, ester or prodrug thereof;
such that the rheumatoid arthritis is treated in the subject.

16. The method of claim 15, wherein R⁴ is hydrogen.

17. A method for treating rheumatoid arthritis in a subject, comprising administering to the subject a tetracycline compound of Formula VI: 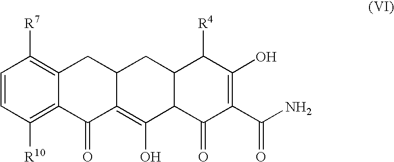
wherein:
R⁴ is amino or hydrogen;
R⁷ amino or hydrogen; and
R¹⁰ is hydrogen, substituted or unsubstituted C₁-C₅ alkyl, substituted or unsubstituted heterocyclyl, substituted or unsubstituted phenyl, substituted or unsubstituted heteroaryl, substituted or unsubstituted acyl, or substituted or unsubstituted imine;
or a pharmaceutically acceptable salt, ester or prodrug thereof;
such that the rheumatoid arthritis is treated in the subject.

18. The method of claim 17, wherein R⁴ is hydrogen and R⁷ is dimethylamino.

19. The method of claim 17, wherein R⁴ is dimethylamino and R⁷ is hydrogen.

20. A method for treating rheumatoid arthritis in a subject, comprising administering to the subject a tetracycline compound selected from the group consisting of: 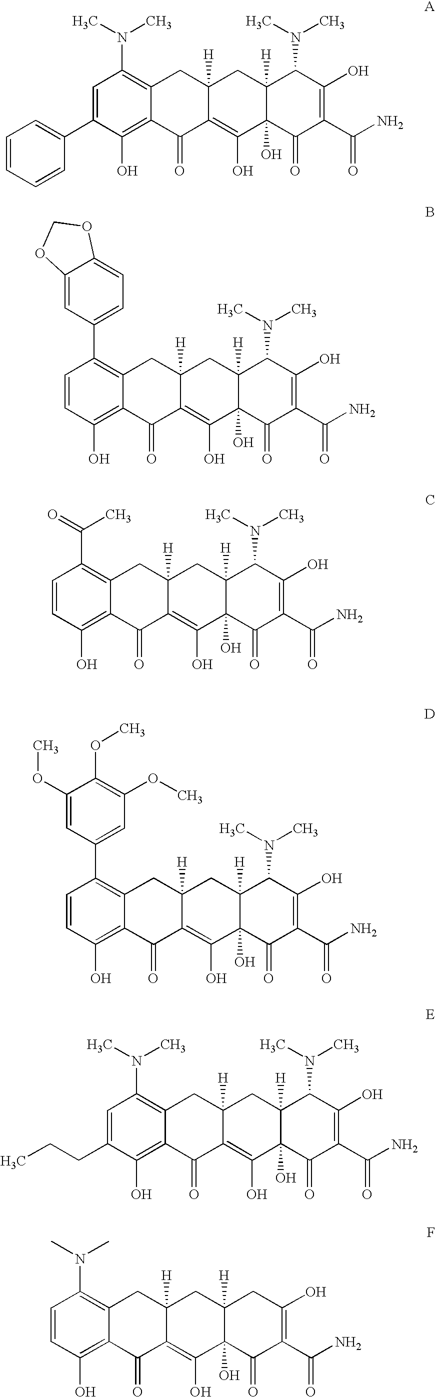 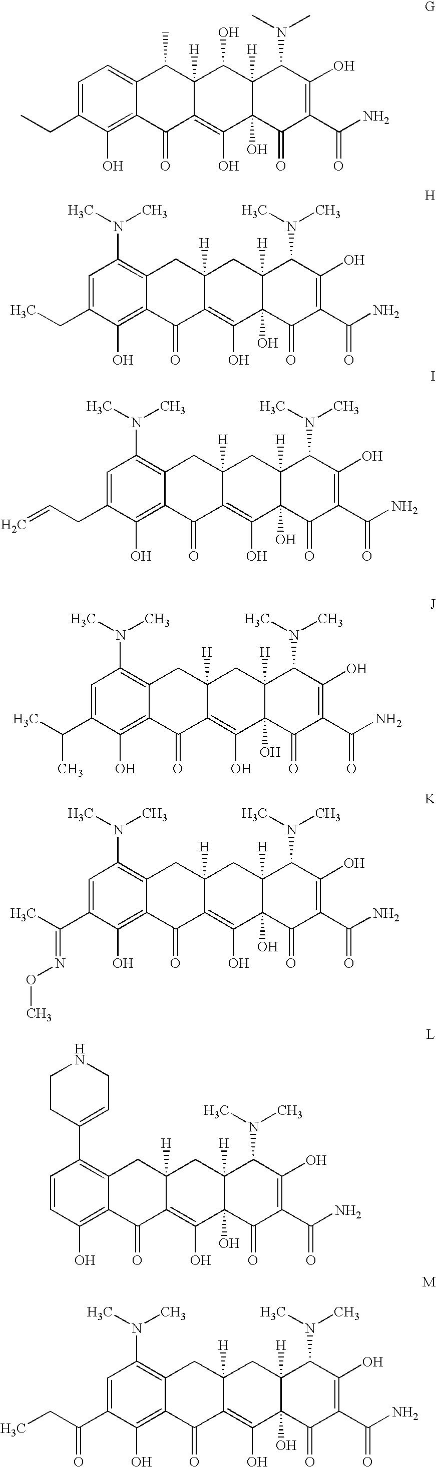 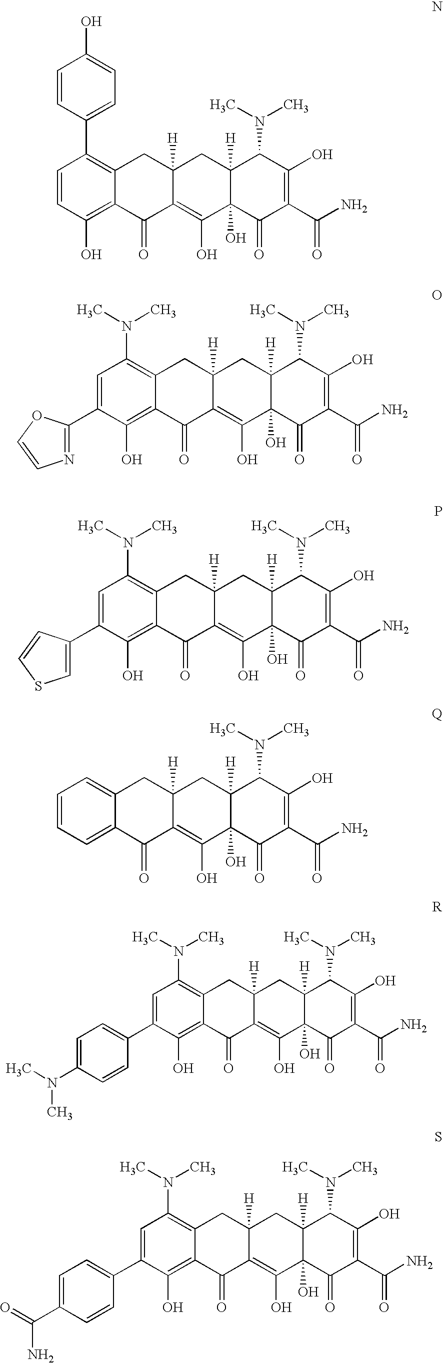 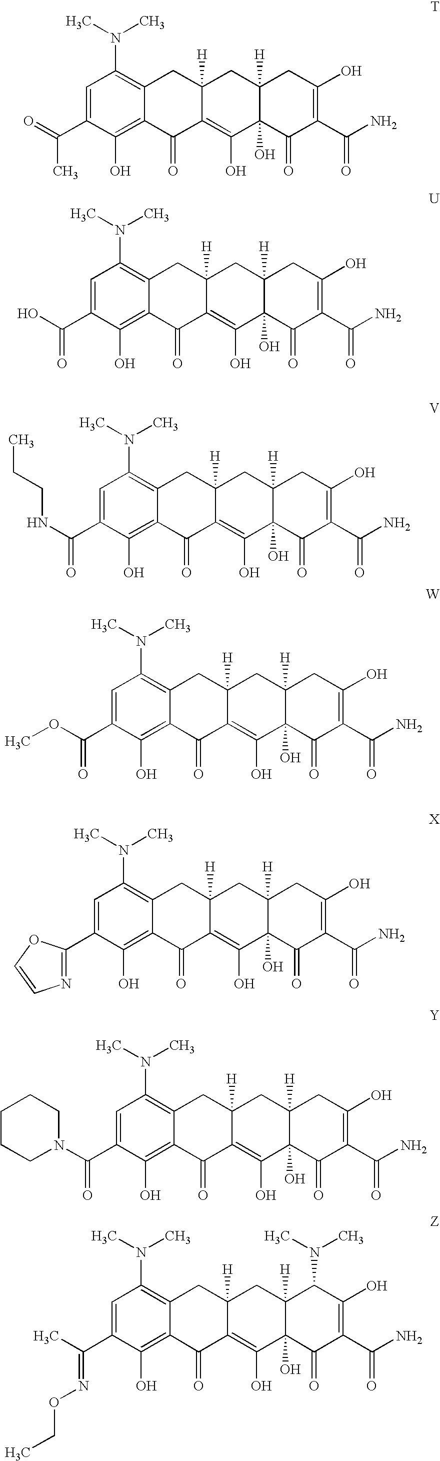 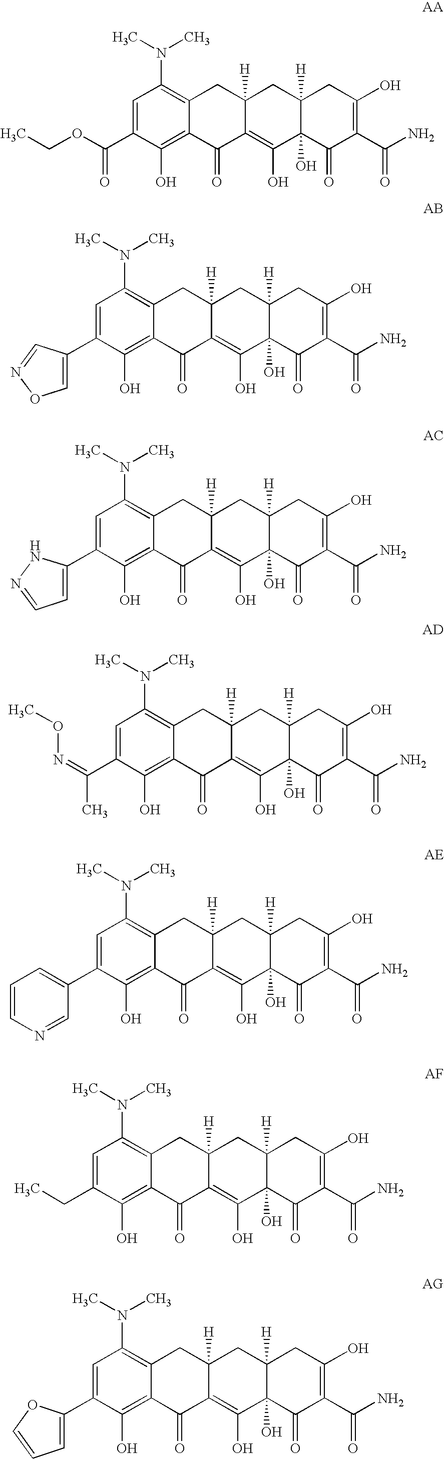 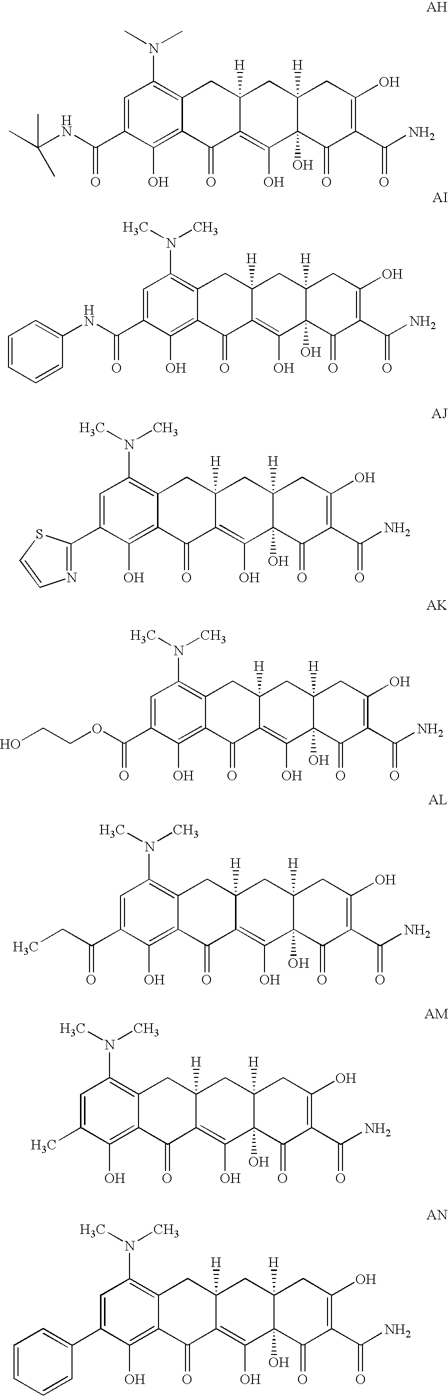 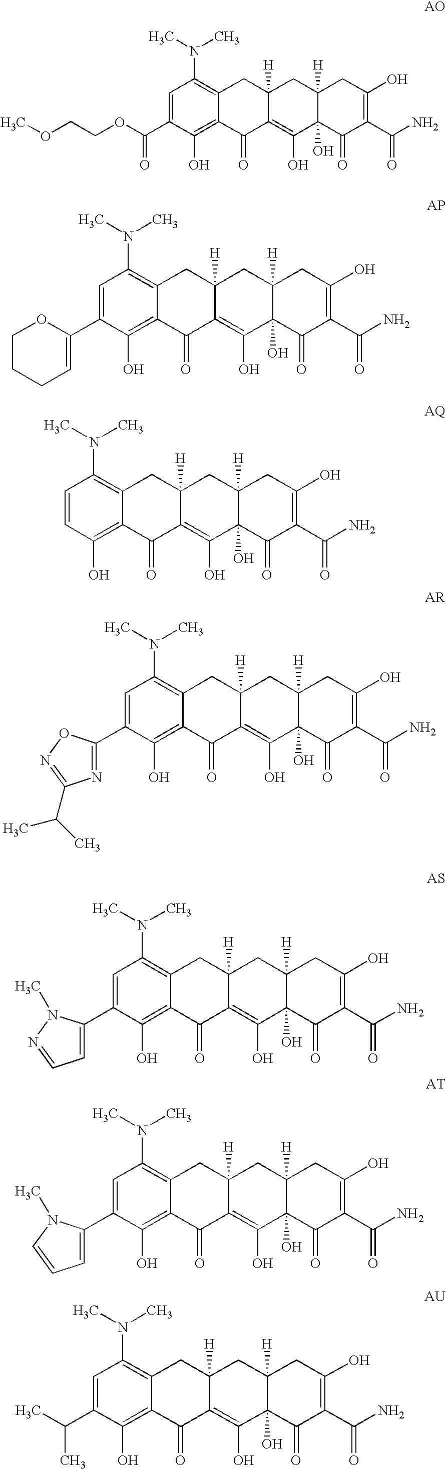 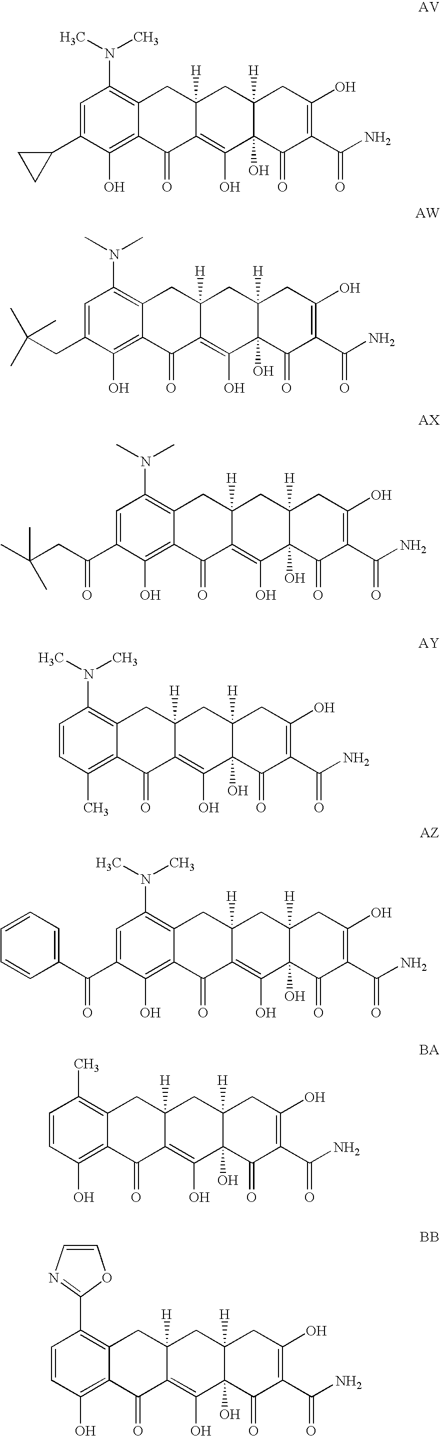 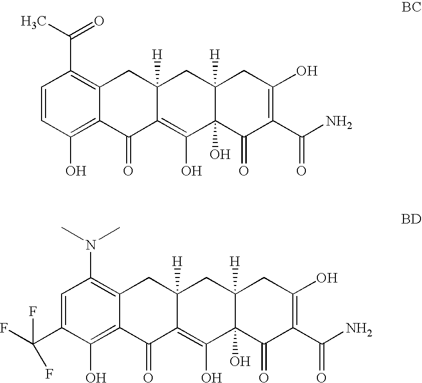
and pharmaceutically acceptable salts, esters and prodrugs thereof, such that the rheumatoid arthritis is treated in the subject.
